(19) United States
(12) Patent Application Publication
(10) Pub. No.: US 2021/0228511 A1
(43) Pub. Date: Jul. 29, 2021

(54) NEURONAL AGONISTS AND ISOTONIC OR HYPERTONIC SALINE FOR TREATING RESPIRATORY DISEASES

(71) Applicant: University of Saskatchewan, Saskatoon (CA)

(72) Inventors: Juan P. Ianowski, Saskatoon (CA); Julian S. Tam, Saskatoon (CA); Xiaojie Luan, Saskatoon (CA); Veronica A. Campanucci, Saskatoon (CA)

(21) Appl. No.: 17/156,167

(22) Filed: Jan. 22, 2021

Related U.S. Application Data
(60) Provisional application No. 62/964,828, filed on Jan. 23, 2020.

(51) Int. Cl. A61K 31/165; A61K 33/14; A61K 9/00; A61K 31/05

(52) U.S. Cl. CPC A61K 31/165; A61K 33/14; A61K 31/05; A61K 9/007; A61K 9/0082

(57) ABSTRACT

The present application relates to a method of increasing the volume of an airway surface liquid (ASL) layer and/or increasing mucociliary clearance in a subject. For example, the application relates the use of one or more neuronal agonists such as menthol and/or capsaicin, in combination with hypertonic saline (HTS) or isotonic saline (ITS) for increasing the volume of airway surface liquid (ASL) and/or increasing mucociliary clearance or for treatment of a disease, disorder or condition treatable by increasing the volume of an ASL layer and/or increasing mucociliary clearance. For example, the disease, disorder or condition is cystic fibrosis or a non-cystic fibrosis respiratory disease, disorder or condition such as non-cystic fibrosis bronchiectasis.

### RELATED APPLICATIONS

The present application claims the benefit of priority of co-pending U.S. provisional patent application No. 62/964,828 filed on Jan. 23, 2020 the contents of which are incorporated herein by reference in their entirety.

### FIELD

The present application relates to a method of increasing the volume of an airway surface liquid (ASL) layer and/or increasing mucociliary clearance in a subject. For example, the application relates the use of one or more neuronal agonists such as menthol or capsaicin or a combination thereof, in combination with hypertonic saline (HTS) treatment or isotonic saline (ITS) treatment for increasing the volume of an airway surface liquid (ASL) layer and/or increasing mucociliary clearance or for treatment of a disease, disorder or condition treatable by increasing the volume of an airway surface liquid (ASL) layer and/or increasing mucociliary clearance. For example, the disease, disorder or condition is a respiratory disease, disorder or condition including cystic fibrosis and a non-cystic fibrosis respiratory disease, disorder or condition such as non-cystic fibrosis bronchiectasis.

### INTRODUCTION

Dehydration of the airway surface liquid layer is believed to contribute to the development of cystic fibrosis (CF) lung disease through defective mucociliary clearance and impaired innate defense against pathogenic organisms. Thus, there has been an interest in developing therapies, such as inhaled hypertonic saline, aimed at restoring airway hydration and increasing mucociliary clearance.

Inhaled hypertonic saline (HTS) is a well-established treatment for patients with cystic fibrosis (CF) and patients with non-CF bronchiectasist². HTS treatment has been shown to improve mucociliary clearance, forced expiratory volume in 1 s, frequency of exacerbations, days on antibiotics, and well-being^{1,2,3,4}. Recent analyses of lung clearance index and spirometry data suggest that HTS treatment may be able to halt the progression of mild CF lung disease⁵. Though CFTR modulators have been shown to improve outcomes in individuals with certain CFTR gene mutations^{6,7,8,9}, HTS is a mutation-agnostic treatment that benefits patients with CF regardless of genotype. However, the clinical benefits from HTS treatment can be modest and have not been demonstrated in all age groups. Moreover, based on clinical experience, not all patients are able to tolerate nebulized HTS due to bronchospasm, cough, and chest discomfort. Thus, there are limits to the therapeutic benefits of HTS nebulization in its current form and further improvements are needed.

The exact mechanism of action of HTS is not understood³, which makes it difficult to develop procedures to improve treatment outcomes such as through modulating the duration and intensity of the treatment effect¹⁰. The current prevalent understanding of the mechanism of action of HTS inhalation is that the treatment generates an osmotic gradient that draws water into the airways^{11,12}. This increases the volume of airway surface liquid (ASL) layer, which improves mucus rheological properties and accelerates mucus transport rates^{3,4}. The intensity of treatment has been proposed to depend on the aquaporin-mediated water permeability of the airway epithelia cells^{11,12}.

However, there have been studies looking into other mechanisms for the mechanism of action of HTS treatment. For example, in rat airways, treatment with HTS was found to stimulate neurogenic inflammation, specifically through the local release of inflammatory mediators by sensory-efferent pathways^{13,14,15}. Additionally, in guinea pig airways, HTS treatment was found to activate airway afferent nerves including Aδ- and C-fibers both in vitro and in vivo^{16,17}. Stimulation of C- and Aδ-fibres may cause local anterograde release (i.e. axon reflex), in addition to reflex mediated by the central nervous system, of neurotransmitters which trigger ASL secretion^{20,21}. Also, treatment of the nasal cavity with HTS in healthy volunteers was found to stimulate nociceptive nerves and glandular mucus exocytosis¹⁸. In addition, HTS induces robust reflex responses in the nose and larynx of guinea pigs, similar to those evoked by capsaicin, and evokes coughing when applied topically to the tracheal or laryngeal mucosa¹⁹. In ferret trachea, HTS stimulates the production of two markers of gland secretion, mucins and lysozyme²².

Inhaled hypertonic saline (HTS) is a well-established treatment to rehydrate the airway in patients with cystic fibrosis (CF)^{2,46}. It has been shown to improve mucociliary clearance, lung function, lung clearance index, and frequency of pulmonary exacerbations in CF patients^{2, 6, 46-50}. Hypertonic saline treatment has the potential to improve the health of CF patients regardless of their genotype, particularly important for those patients with CFTR mutations for which there are no effective CFTR modulators^{7-9,51}. However, the clinical benefits from HTS treatment can be modest and have not been demonstrated in all age groups⁵⁰. Moreover, based on clinical experience, not all patients are able to tolerate nebulized HTS due to bronchospasm, cough, and chest discomfort. Thus, there are limits to the therapeutic benefits of HTS nebulization in its current form.

A number of clinical trials are aimed at developing novel airway rehydration therapies. However, most efforts concentrate on developing epithelial sodium channel (ENaC) blockers to inhibit airway surface fluid reabsorption (see, for example, clinical trials for Parion PS-G201; Boehringer Ingelheim B11265162; AstraZeneca AZD5643; Novartis QBW276; Spyryx Biosciences SPX101). None of the current efforts concentrate on developing strategies of stimulating the sensory fibers innervating the lung to increase the HTS-triggered fluid secretion.

### SUMMARY

The Applicant has found that hypertonic saline (HTS) treatment causes ASL production in part through the stimulation of the nervous system by, for example, triggering active ASL secretion by airway epithelia, and that this neuronal stimulation pathway occurs alongside the osmotic effect of HTS that draws water from the serosal surface into the ASL. The Applicant has further surprisingly found that it is possible to increase the height (volume) of the ASL, increase mucociliary clearance and/or increase the duration and intensity of HTS treatment or isotonic saline (ITS) treatment using one or more neuronal agonists in combination with HTS treatment or ITS treatment.

Accordingly, the present application includes a method of increasing the volume of an airway surface liquid (ASL) layer comprising administering an effective amount of one or more neuronal agonists or a pharmaceutically acceptable salt, prodrug and/or solvate thereof, in combination with hypertonic saline (HTS) treatment or isotonic saline (ITS) treatment, to a subject in need thereof.

The present application also includes a method of treating a disease, disorder or condition that is treatable by increasing the volume of ASL comprising administering an effective amount of one or more neuronal agonists or a pharmaceutically acceptable salt, prodrug and/or solvate thereof, in combination with HTS treatment or ITS treatment, to a subject in need thereof.

The present application further includes a method of enhancing hydration of an airway surface comprising administering an effective amount of one or more neuronal agonists or a pharmaceutically acceptable salt, prodrug and/or solvate thereof, in combination with HTS treatment or ITS treatment, to a subject in need thereof.

The present application also includes a method of treating a disease, disorder or condition that is treatable by enhancing hydration of an airway surface comprising administering an effective amount of one or more neuronal agonists or a pharmaceutically acceptable salt, prodrug and/or solvate thereof, in combination with HTS treatment or ITS treatment, to a subject in need thereof.

The present application further includes a method of increasing airway clearance comprising administering an effective mount of one or more neuronal agonists, or a pharmaceutically acceptable salt, prodrug and/or solvate thereof, in combination with HTS treatment or ITS treatment, to a subject in need thereof.

The present application also includes a method of treating a disease, disorder or condition that is treatable by increasing airway clearance comprising administering an effective amount of one or more neuronal agonists, or a pharmaceutically acceptable salt, prodrug and/or solvate thereof, in combination with HTS treatment or ITS treatment, to a subject in need thereof.

The present application further includes a method of improving the efficacy of HTS treatment comprising administering an effective amount of one or more neuronal agonists or a pharmaceutically acceptable salt, prodrug and/or solvate thereof, in combination with the HTS treatment or ITS treatment, to a subject in need thereof.

The present application further includes a composition comprising one or more neuronal agonists, or a pharmaceutically acceptable salt, prodrug and/or solvate thereof, and HTS and/or ITS, wherein the one or more neuronal agonists, or a pharmaceutically acceptable salt, prodrug and/or solvate thereof, and HTS are present in amounts effective to increase the volume of ASL and/or for treating a disease, disorder or condition treatable by increasing the volume of ASL.

The present application also includes a composition comprising one or more neuronal agonists, or a pharmaceutically acceptable salt, prodrug and/or solvate thereof, and HTS and/or ITS, wherein the one or more neuronal agonists, or a pharmaceutically acceptable salt, prodrug and/or solvate thereof, and HTS and/or ITS are present in amounts effective to enhance hydration of an airway surface or for treating a disease, disorder or condition treatable by enhancing hydration of an airway surface.

The present application further includes a composition comprising one or more neuronal agonists, or a pharmaceutically acceptable salt, prodrug and/or solvate thereof, and HTS and/or ITS, wherein the one or more neuronal agonists, or a pharmaceutically acceptable salt, prodrug and/or solvate thereof, and HTS are present in amounts effective to increase mucociliary clearance or for treating a disease, disorder or condition treatable by increasing mucociliary clearance.

The present application also includes a composition comprising one or more neuronal agonists, or a pharmaceutically acceptable salt, prodrug and/or solvate thereof, and HTS or ITS, wherein the one or more neuronal agonists, or a pharmaceutically acceptable salt, prodrug and/or solvate thereof, and HTS or ITS are present in amounts effective to improve the efficacy of HTS treatment.

The present application further includes a kit for increasing the volume of ASL or for treating a disease, disorder or condition treatable by increasing the volume of ASL comprising one or more neuronal agonists, or a pharmaceutically acceptable salt, prodrug and/or solvate thereof, and HTS or ITS and optionally instructions for administration of the one or more neuronal agonists, or a pharmaceutically acceptable salt, prodrug and/or solvate thereof, in combination with the HTS or ITS.

The present application further includes a kit for enhancing hydration of an airway surface or for treating a disease, disorder or condition treatable by enhancing hydration of an airway surface comprising one or more neuronal agonists, or a pharmaceutically acceptable salt, prodrug and/or solvate thereof, and HTS or ITS and optionally instructions for administration of the one or more neuronal agonists, or a pharmaceutically acceptable salt, prodrug and/or solvate thereof, in combination with the HTS or ITS.

The present application further includes a kit for increasing airway clearance or for treating a disease, disorder or condition treatable by increasing airway clearance comprising one or more neuronal agonists, or a pharmaceutically acceptable salt, prodrug and/or solvate thereof, and HTS or ITS and optionally instructions for administration of the one or more neuronal agonists, or a pharmaceutically acceptable salt, prodrug and/or solvate thereof, in combination with the HTS or ITS.

The present application also includes a kit for improving the efficacy of HTS treatment or ITS treatment comprising one or more neuronal agonists, or a pharmaceutically acceptable salt, prodrug and/or solvate thereof, and HTS or ITS and optionally instructions for administration of the one or more neuronal agonists, or a pharmaceutically acceptable salt, prodrug and/or solvate thereof, in combination with the HTS or ITS.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows an experimental design and phase contrast imaging using synchrotron x-rays. A is a schematic showing the set-up for airway surface liquid (ASL) layer height measurement in the lumen of the trachea using phase contrast imaging. When x-rays pass through the preparation, the difference in refractive index between the ASL and the air results in a phase shift of x-rays that causes a distinct interference pattern detected as variations in x-ray intensities on the CCD (see ref 23 for ex vivo diagram). B shows synchrotron-based phase contrast imaging measurement of ASL height in an isolated swine trachea. C is a graph showing hypertonic saline (HTS) or isotonic saline (ITS) aerosol were delivered at time 0 for 90 seconds, and images were acquired at time −3, 6, 12, and 18 minutes. D and E are representative sample of the images acquired from an ex vivo preparation treated with (1D) HTS and (1E) ITS nebulization at −3, 6, 12, and 18 min.

FIG. 2 contains graphs showing that HTS triggers ASL secretion in vivo and ex vivo preparations. A is a scatter plot of HTS and ITS treatment on ASL height in live swine. B is a graph showing change in ASL height (HTS, n=6 beads from 4 swine; ITS, n=6 beads from 5 swine). C is scatter plot of HTS and ITS treatment on ASL volume in ex vivo trachea preparation. D is a graph showing change in ASL height (HTS, n=45 beads from 15 tracheas; ITS, n=49 beads from 14 tracheas; control, n=12 beads from 5 tracheas). E is a graph showing amiloride did not affect the HTS treatment result (HTS, n=45 beads from 15 tracheas; ITS, n=49 beads from 14 tracheas; HTS+Amil, n=12 beads from 5 tracheas). Data are presented as mean±SEM and values at 18 min were analyzed with ANOVA and Tukey's multiple comparison test. Data sets labeled with different letters differ significantly, p<0.05.

FIG. 3 contains graphs showing that the nervous system contributes to HTS-triggered ASL secretion. A is a graph showing atropine (atro) combined with lidocaine (lido) reduced HTS-triggered ASL secretion but had no effect in ITS-treated swine in vivo (HTS, n=6 beads from 4 animals; ITS, n=6 beads from 5 animals; HTS+Atro+Lido, n=6 beads from 5 animals; ITS+Atro+Lido, n=5 beads from 4 animals). B is a graph showing that stimulating C-fibers with capsaicin increased the secretion during ITS and HTS treatment in live swine (HTS, n=6 beads from 4 animals; ITS, n=6 beads from 5 animals; HTS+Capsaicin, n=6 beads from 4 animals; ITS+Capsaicin, n=5 beads from 4 animals). Data are presented as mean±SEM and values at 18 min were analyzed with ANOVA and Tukey's multiple test. Data sets labeled with different letters differ significantly, p<0.05.

FIG. 4 are graphs showing that HTS stimulates ex vivo ASL secretion via activation of sensory neurons and release of acetylcholine in wildtype and CF airways. A is a graph showing treatment with lidocaine (Lido, 80 mg/ml aerosol) plus tetrodotoxin (TTX, 1 μM) (HTS, n=45 beads from 15 tracheas; ITS, n=49 beads from 14 tracheas; HTS+Lido+TTX, n=18 beads from 4 tracheas; ITS+Lido+TTX, n=9 beads from 4 tracheas). B is a graph showing Incubation with CFTRinh172 (172, 100 μM) (HTS, n=45 beads from 15 tracheas; ITS, n=49 beads from 14 tracheas; HTS+172, n=59 beads from 12 tracheas; ITS+172, n=29 beads from 8 tracheas). C is a graph showing Incubation with lidocaine plus TTX on CFTRinh172-treated airway (HTS+172, n=59 beads from 12 tracheas; HTS+172+Lido+TTX, n=9 beads from 4 tracheas; ITS+172, n=29 beads from 8 tracheas; ITS+172+Lido+TTX, n=21 beads from 6 tracheas). D is a graph showing the effect of L-703606 (NK-1 blocker, 1 μM) (HTS, n=45 beads from 15 tracheas; ITS, n=49 beads from 14 tracheas; HTS+NK-1 blocker, n=36 beads from 12 tracheas; ITS+NK-1 blocker, n=16 beads from 5 tracheas). E is a graph showing the effect of L-703606 on CFTRinh172-treated preparations (HTS+172, n=59 beads from 12 tracheas; HTS+172+NK-1 blocker, n=10 beads from 4 tracheas; ITS+172, n=29 beads from 8 tracheas; ITS+172+NK-1 blocker, n=17 beads from 6 tracheas). F is a graph showing the effect of atropine (1 μM) (HTS, n=45 beads from 15 tracheas; ITS, n=49 beads from 14 tracheas; HTS+Atropine, n=22 beads from 5 tracheas; ITS+Atropine, n=11 beads from 4 tracheas). G is a graph showing the effect of atropine (1 μM) on CFTRinh172-treated preparations (HTS+172, n=59 beads from 12 tracheas; HTS+172+Atropine, n=18 beads from 5 tracheas; ITS+172, n=29 beads from 8 tracheas; ITS+172+Atropine, n=19 beads from 5 tracheas). H is a graph showing the effect of lidocaine, TTX, and atropine on CFTR−/− swine trachea (HTS, n=6 beads from 2 tracheas; HTS+Lido+TTX+Atro, n=4 beads from 2 tracheas; ITS, n=4 beads from 2 tracheas). Data are presented as mean±SEM and values at 18 min were analyzed with ANOVA and Tukey's multiple test. Data sets labeled with different letters differ significantly, p<0.05.

FIG. 5 contains graphs showing HTS treatments stimulate active ASL production by airway epithelia. A is a graph showing treatment with the CFTR blocker CFTRinh172 (100 μM) reduced HTS-triggered ASL height increase, and treatment with CFTRinh172, bumetanide (100 μM), and niflumic acid (100 μM) in HC₃⁻-free saline solution bath reduced HTS-triggered ASL height increase even further (HTS, n=45 beads from 15 tracheas; HTS+172, n=59 beads from 12 tracheas; HTS+Bumet+NA+172+HCO₃⁻-free, n=24 beads from 7 tracheas). B is a graph that shows that in ITS-treated preparations incubation with the ion transport blocker cocktail (CFTRinh172, bumetanide, and niflumic acid in HCO₃⁻-free bath) had a similar effect as CFTRinh172 alone (ITS, n=49 beads from 14 tracheas; ITS+172, n=29 beads from 8 tracheas; ITS+Bumet+NA+172+HCO₃⁻-free, n=18 beads from 7 tracheas). C is a graph that shows approximately 50% of the ASL produced by HTS in airways without CFTR function is the result of the osmotic effect. After blocking all ion transport with CFTRinh172, bumetanide, and niflumic acid in HCO₃⁻-free saline, HTS produced ˜50% less ASL secretion than that produced by preparations incubated with CFTRinh172 alone (HTS+172, n=59 beads from 12 tracheas; HTS+Bumet+NA+172+HCO₃⁻-free, n=24 beads from 7 tracheas; ITS+Bumet+NA+172+HCO₃⁻-free, n=18 beads from 7 tracheas). Data are presented as mean±SEM and values at 18 min were analyzed with ANOVA and Tukey's multiple comparison test. Data sets labeled with different letters differ significantly, p<0.05.

FIG. 6 shows tantalum particle movement. FIG. 6 is composed of two consecutive images merged. The change in X position and change in Y position were used to calculate the hypotenuse of an individual particle's movement between consecutive images. The hypotenuse was considered to be the distance travelled in that 30-second interval.

FIG. 7 contains graphs showing that the increase in ASL volume induced by HTS peaks at ˜90 min after treatment in wild-type swine trachea preparations (ex vivo, A) as well as in living swine (in vivo, B).

FIG. 8 is a graph showing that the use of ion channel blockers and CFTR blocker CFTRinh172 (172) decreases the effect of HTS on ASL secretion in wild-type swine trachea preparations (ex vivo).

FIG. 9 is a graph showing that ASL secretion from HTS treatment peaks earlier in cystic fibrosis pigs (CFTR knockout) than in wild-type pig, and that less fluid is produced.

FIG. 10 contains graphs showing that adding exemplary neuronal agonists of the application capsaicin (cap) and menthol (ment) to HTS or ITS increases the volume of fluid produced and prolongs the duration of action of HTS or ITS in wild-type swine in vivo (A) and of HTS in CF swine (B) in vivo.

FIG. 11 is a graph showing that adding exemplary neuronal agonist, menthol alone to HTS increases the intensity and duration of treatment in WT (wild-type) swine in vivo but to a lesser extent than in conjunction with exemplary neuronal agonist capsaicin

FIG. 12 is a graph showing that exemplary neuronal agonists capsaicin and menthol stimulate active fluid secretion by airway submucosal glands in WT swine.

FIG. 13 is a graph showing that in WT swine airways, HTS-triggered or ITS-triggered increase in mucociliary clearance is augmented by the addition of exemplary neuronal agonist capsaicin.

FIG. 14 is a graph showing mucociliary clearance in ex vivo swine trachea preparations following treatment with HTS plus exemplary neuronal agonists capsaicin and menthol. The mucociliary clearance mean speed was measured immediately after HTS nebulization and 90 min, and 150 min after treatment. Addition of exemplary neuronal agonists capsaicin and menthol to the HTS formulation extended the duration of HTS-triggered mucociliary clearance by at least 60 min.

FIG. 15 is a graph showing mucociliary clearance in ex vivo swine trachea preparations following treatment with HTS plus exemplary neuronal agonist isopulegol. The mucociliary clearance mean speed was measured immediately after HTS nebulization and 90 min, and 150 min after treatment. Addition of exemplary neuronal agonist (−)-isopulegol to the HTS formulation extended the duration of HTS-triggered mucociliary clearance.

### DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise. In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in this application, the singular forms “a”, “an” and “the” include plural references unless the content clearly dictates otherwise. For example, an embodiment including “a compound” should be understood to present certain aspects with compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “solvate” as used herein means a compound, or a salt or prodrug of a compound, wherein molecules of a suitable solvent are incorporated in the crystal lattice.

The term “pharmaceutically acceptable” means compatible with the treatment of subjects.

The term “carrier” as used herein means an inert compound with which the composition is mixed or formulated. The term “carrier” includes, for example, solid or liquid carriers or combinations thereof.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice.

The term “prodrug” as used herein means a compound, or salt and/or solvate of a compound, that, after administration, is converted into an active drug.

The term “subject” as used herein includes all members of the animal kingdom including mammals, and suitably refers to humans. Thus the methods and uses of the present application are applicable to both human therapy and veterinary applications.

The term “administered” as used herein means administration of a therapeutically effective amount of one or more compounds or compositions to a cell, tissue, organ or subject.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of one or more of compounds or compositions and optionally consist of a single administration, or alternatively comprise a series of administrations.

As used herein, the term “effective amount” or “therapeutically effective amount” means an amount effective, at dosages and for periods of time necessary to achieve a desired result. The terms “to treat”, “treating” and “treatment” as used herein and as is well understood in the art, means an approach for obtaining beneficial or desired results, including clinical results.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The term “disease, disorder or condition” as used herein refers to a disease, disorder or condition treatable by hypertonic saline (HTS) or isotonic saline (ITS).

The term “non-cystic fibrosis respiratory disease, disorder or conditions” as used herein refers to a disease, disorder or condition characterized by an accumulation of and/or a difficulty in clearing of airway secretions, not including cystic fibrosis.

The term “treatable by increasing the volume of airway surface liquid (ASL) layer” as used herein means that the disease, disorder or condition to be treated is affected by, modulated by and/or has some biological basis, either direct or indirect, that includes an airway surface liquid (ASL) layer.

The term “treatable by increasing airway clearance” as used herein means that the disease, disorder or condition to be treated is affected by, modulated by and/or has some biological basis, either direct or indirect, that includes airway clearance.

The term “treatable by increasing mucociliary clearance” as used herein means that the disease, disorder or condition to be treated is affected by, modulated by and/or has some biological basis, either direct or indirect, that includes mucociliary clearance.

The term “saline” as used herein refers to a solution comprising sodium chloride in water.

The term “hypertonic saline” or “HTS” as used herein refers to a solution of sodium chloride in water having a concentration of sodium chloride greater than 0.9% w/v.

The term “hypertonic saline treatment” or “HTS treatment” as used herein refers to methods of administering an effective amount of HTS to a subject in need of such treatment, which is generally via inhalation using a nebulizer.

The term “isotonic saline” or “ITS” as used herein refers to a solution of sodium chloride in water having a concentration of sodium chloride of 0.9% w/v.

The term “isotonic saline treatment” or “ITS treatment” as used herein refers to methods of administering an effective amount of ITS to a subject in need of such treatment, which is generally via inhalation using a nebulizer.

An “aerosol,” as used herein refers to a continuous gas phase and, dispersed therein, a discontinuous phase of liquid and/or solid particles.

The term “nebulization” or “nebulized” as used herein, refers to the conversion of a liquid, such as a liquid solution, emulsion, or suspension, into an aerosol. For example, a nebulized aerosol comprises liquid droplets dispersed in a continuous gas phase. The liquid droplet may optionally comprise solid particles which are suspended within the droplets.

A “nebulizer,” as used herein, is a device which is capable of converting a liquid material into a nebulized aerosol which is typically inhalable by a human via the nose or the mouth into the lungs.

The term “neuronal agonist” as used herein refers to a compound that binds to a neuronal receptor in the respiratory system of a subject and stimulates the neuronal receptor activity.

The term “menthol” as used herein refers to a compound having the IUPAC name: 5-Methyl-2-(propan-2-yl)cyclohexan-1-ol or 2-Isopropyl-5-methylcyclohexan-1-ol, and having the chemical formula:

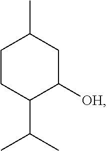

and all stereoisomers thereof.

The term “isopulegol” as used herein refers to a compound having the IUPAC name: 5-methyl-2-prop-1-en-2-ylcyclohexan-1-ol, and having the chemical formula:

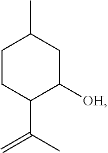

and all stereoisomers thereof.

The term “capsaicin” as used herein refers to a compound having the IUPAC name: (6E)-N-[(4-Hydroxy-3-methoxyphenyl)methyl]-8-methylnon-6-enamide or (E)-N-(4-Hydroxy-3-methoxybenzyl)-8-methylnon-6-enamide or 8-Methyl-N-vanillyl-trans-6-nonenamide and having the chemical formula:

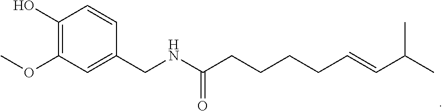 [SMILES: COc1cc(CNC(=O)CCCC/C=C/C(C)C)ccc1O]

The term “olvanil” as used herein refers to a compound having the IUPAC name: (Z)-N-[(4-hydroxy-3-methoxyphenyl)methyl]octadec-9-enamide or N-vanillyloleamide or N-vannilyloleoylamide and having the chemical formula:

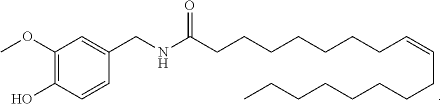 [SMILES: CCCCCCCC/C=C\CCCCCCCC(=O)NCc1ccc(O)c(OC)c1]

The term “arvanil” as used herein refers to a compound having the IUPAC name: (5Z,8Z,11Z,14Z)-N-[(4-hydroxy-3-methoxyphenyl)methyl]icosa-5,8,11,14-tetraenamide, or N-vanillylarachidonamide and having the chemical formula:

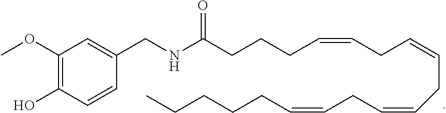 [SMILES: CCCCC/C=C\C/C=C\C/C=C\C/C=C\CCCC(=O)NCc1ccc(O)c(OC)c1]

The terms “airway surface” as used herein refers to the surface of any portion of the respiratory system of a subject.

The term “airway surface liquid layer” or “ASL layer” as used herein refers to a layer of fluid or liquid covering a surface in the respiratory system.

The term “hydrated” or “hydration” as used herein refers to bringing, placing, and/or drawing (and/or the like) water onto an airway surface.

The term “mucociliary clearance” or “MCC” as used herein refers to the movement and/or removal of particles from the airway secretion through the action of cilia including, for example, mucus from the respiratory system.

The term “airway clearance” as used herein refers to the movement and/or removal of airway secretion including mucus from the respiratory system by any mechanism known to move and/or remove particles including, for example, coughing and/or physiotherapy such as chest physiotherapy.

### II. Methods and Uses of the Application

The Applicant has unexpectedly shown that HTS-stimulated ASL layer height increase in both wild-type and CFTR−/− swine is reduced by inhibiting either neuronal function or epithelial ion secretion into the ASL. Accordingly, it has been surprisingly found that HTS treatment causes ASL production in part through the stimulation of the nervous system by, for example, triggering active ASL secretion by airway epithelia, and that this neuronal stimulation pathway occurs alongside the osmotic effect of HTS that draws water from the serosal surface into the ASL layer.

The Applicant has also unexpectedly shown that the use of one or more neuronal agonists, for example, menthol, capsaicin, or isopulegol and mixtures thereof, in combination with HTS treatment results in a greater increase in the height (volume) of the airway surface liquid (ASL) layer in wild type (WT) and cystic fibrosis (CF) swine in vivo compared to the increase in the height (volume) of the ASL layer when using HTS treatment alone. It was further shown that the use of one or more neuronal agonists, for example, menthol, capsaicin, or isopulegol in combination with HTS treatment resulted in greater mucociliary clearance compared to using HTS treatment alone. Accordingly, the Applicant has further surprisingly found that it is possible to increase in the height (volume) of the ASL layer, increase mucociliary clearance and increase the duration and intensity of HTS treatment by pharmacologically modulating the contribution of the nervous system to HTS-triggered ASL secretion using one or more neuronal agonists in combination with HTS treatment.

The use of one or more neuronal agonists in combination with ITS treatment was also found to increase the height (volume) of the airway surface liquid (ASL) layer and to increase mucociliary clearance in wild type (WT) swine in vivo compared to the increase in the height (volume) of the ASL layer or mucociliary clearance when using isotonic saline (ITS) treatment alone. Accordingly, the Applicant has further found that it is possible to increase in the height (volume) of the ASL layer, increase mucociliary clearance and increase the duration and intensity of ITS treatment by pharmacologically using one or more neuronal agonists in combination with ITS treatment.

Accordingly, the present application includes a method of increasing the volume of an airway surface liquid (ASL) layer comprising administering an effective amount of one or more neuronal agonists or a pharmaceutically acceptable salt, prodrug and/or solvate thereof, in combination with hypertonic saline (HTS) treatment or isotonic saline (ITS) treatment, to a subject in need thereof.

The present application also includes a method of treating a disease, disorder or condition that is treatable by increasing the volume of an ASL layer comprising administering an effective amount of one or more neuronal agonists or a pharmaceutically acceptable salt, prodrug and/or solvate thereof, in combination with HTS treatment or ITS treatment, to a subject in need thereof.

The present application also includes a use of one or more neuronal agonists or a pharmaceutically acceptable salt, prodrug and/or solvate thereof in combination with HTS treatment or ITS treatment for increasing the volume of an ASL layer or for treating a disease, disorder or condition treatable by increasing the volume of an ASL layer, as well as a use of one or more neuronal agonists or a pharmaceutically acceptable salt, prodrug and/or solvate thereof, in combination with HTS treatment or ITS treatment, for the preparation of a medicament for increasing the volume of an ASL layer or for treating a disease, disorder or condition treatable by increasing the volume of an ASL layer. The application further includes one or more neuronal agonists or a pharmaceutically acceptable salt, prodrug and/or solvate thereof, in combination with HTS treatment or ITS treatment, for use in increasing the volume of an ASL layer, or for treating a disease, disorder or condition treatable by increasing the volume of ASL layer. In an embodiment, the one or more neuronal agonists or a pharmaceutically acceptable salt, prodrug and/or solvate thereof, are administered or used in combination with HTS treatment for increasing the volume of an ASL layer or for treating a disease, disorder or condition treatable by increasing the volume of an ASL layer.

In an embodiment, the one or more neuronal agonists or a pharmaceutically acceptable salt, prodrug and/or solvate thereof, are administered or used in combination with ITS treatment for increasing the volume of an ASL layer or for treating a disease, disorder or condition treatable by increasing the volume of an ASL layer.

Increasing the height (volume) of an ASL layer and/or increasing ASL secretion generally improves hydration of the airway surface.

Accordingly, the present application includes a method of enhancing hydration of an airway surface comprising administering an effective amount of one or more neuronal agonists or a pharmaceutically acceptable salt, prodrug and/or solvate thereof, in combination with HTS treatment or ITS treatment, to a subject in need thereof.

The present application also includes a method of treating a disease, disorder or condition that is treatable by enhancing hydration of an airway surface comprising administering an effective amount of one or more neuronal agonists or a pharmaceutically acceptable salt, prodrug and/or solvate thereof, in combination with HTS treatment or ITS treatment, to a subject in need thereof.

The present application also includes a use of one or more neuronal agonists or a pharmaceutically acceptable salt, prodrug and/or solvate thereof, in combination with HTS treatment or ITS treatment, for enhancing hydration of an airway surface or for treating a disease, disorder or condition treatable by enhancing hydration of an airway surface, as well as a use of one or more neuronal agonists, or a pharmaceutically acceptable salt, prodrug and/or solvate thereof, in combination with HTS treatment or ITS treatment, for the preparation of a medicament for enhancing hydration of an airway surface or for treating a disease, disorder or condition treatable by enhancing hydration of an airway surface. The application further includes one or more neuronal agonists or a pharmaceutically acceptable salt, prodrug and/or solvate thereof, in combination with HTS treatment or ITS treatment, for use in enhancing hydration of an airway surface or for treating a disease, disorder or condition treatable by enhancing hydration of an airway surface.

In an embodiment, the one or more neuronal agonists or a pharmaceutically acceptable salt, prodrug and/or solvate thereof, are administered or used in combination with HTS treatment for enhancing hydration of an airway surface or for treating a disease, disorder or condition treatable by enhancing hydration of an airway surface.

In an embodiment, the one or more neuronal agonists or a pharmaceutically acceptable salt, prodrug and/or solvate thereof, are administered or used in combination with ITS treatment for enhancing hydration of an airway surface or for treating a disease, disorder or condition treatable by enhancing hydration of an airway surface.

The Applicant has shown that the use of one or more neuronal agonists improves airway clearance, in particular, mucociliary clearance in combination with HTS treatment or ITS treatment in wild type swine.

Accordingly, in an embodiment, the present application includes a method of increasing airway clearance comprising administering an effective mount of one or more neuronal agonists, or a pharmaceutically acceptable salt, prodrug and/or solvate thereof, in combination with HTS treatment or ITS treatment, to a subject in need thereof.

The present application also includes a method of treating a disease, disorder or condition that is treatable by increasing airway clearance comprising administering an effective amount of one or more neuronal agonists, or a pharmaceutically acceptable salt, prodrug and/or solvate thereof, in combination with HTS treatment or ITS treatment, to a subject in need thereof.

The present application also includes a use of one or more neuronal agonists, or a pharmaceutically acceptable salt, prodrug and/or solvate thereof in combination with HTS treatment or ITS treatment, for increasing airway clearance or for treating a disease, disorder or condition treatable by increasing airway clearance, as well as a use of one or more neuronal agonists, or a pharmaceutically acceptable salt, prodrug and/or solvate thereof, in combination with HTS treatment or ITS treatment for the preparation of a medicament for increasing airway clearance or for treating a disease, disorder or condition treatable by increasing airway clearance. The application further includes one or more neuronal agonists, or a pharmaceutically acceptable salt, prodrug and/or solvate thereof, in combination with HTS treatment or ITS treatment for use in increasing airway clearance or for treating a disease, disorder or condition treatable by increasing airway clearance.

In an embodiment, the one or more neuronal agonists or a pharmaceutically acceptable salt, prodrug and/or solvate thereof, are administered or used in combination with HTS treatment for increasing airway clearance or for treating a disease, disorder or condition treatable by increasing airway clearance.

In an embodiment, the one or more neuronal agonists or a pharmaceutically acceptable salt, prodrug and/or solvate thereof, are administered or used in combination with ITS treatment for increasing airway clearance or for treating a disease, disorder or condition treatable by increasing airway clearance.

The present application also includes a method increasing mucociliary clearance comprising administering an effective amount of one or more neuronal agonists, or a pharmaceutically acceptable salt, prodrug and/or solvate thereof, in combination with HTS treatment or ITS treatment, to a subject in need thereof.

The present application also includes a method of treating a disease, disorder or condition that is treatable by increasing mucociliary clearance comprising administering an effective amount of one or more neuronal agonists, or a pharmaceutically acceptable salt, prodrug and/or solvate thereof, in combination with HTS treatment or ITS treatment, to a subject in need thereof.

The present application also includes a use of one or more neuronal agonists, or a pharmaceutically acceptable salt, prodrug and/or solvate thereof in combination with HTS treatment or ITS treatment, for increasing mucociliary clearance or for treating a disease, disorder or condition treatable by increasing mucociliary clearance, as well as a use of one or more neuronal agonists, or a pharmaceutically acceptable salt, prodrug and/or solvate thereof, in combination with HTS treatment or ITS treatment for the preparation of a medicament for increasing mucociliary clearance or for treating a disease, disorder or condition treatable by increasing mucociliary clearance. The application further includes one or more neuronal agonists, or a pharmaceutically acceptable salt, prodrug and/or solvate thereof, in combination with HTS treatment or ITS treatment for use in increasing mucociliary clearance or for treating a disease, disorder or condition treatable by increasing mucociliary clearance.

In an embodiment, the one or more neuronal agonists or a pharmaceutically acceptable salt, prodrug and/or solvate thereof, are administered or used in combination with HTS treatment for increasing mucociliary clearance or for treating a disease, disorder or condition treatable by increasing mucociliary clearance.

In an embodiment, the one or more neuronal agonists or a pharmaceutically acceptable salt, prodrug and/or solvate thereof, are administered or used in combination with ITS treatment for increasing mucociliary clearance or for treating a disease, disorder or condition treatable by increasing mucociliary clearance.

In an embodiment, the increasing airway clearance is by increasing mucociliary clearance.

In an embodiment, the airway surface includes bronchi, bronchioles, alveolar surfaces, and/or nasal and sinus surfaces.

The Applicant has shown that the use of one or more neuronal agonists in combination with HTS treatment or ITS treatment increases the duration and/or intensity of the effects of the HTS treatment or ITS treatment by, for example, increasing HTS-triggered or ITS-triggered ASL secretion and/or mucociliary clearance.

Accordingly, the present application also includes a method of improving the efficacy of HTS treatment or ITS treatment comprising administering an effective amount of one or more neuronal agonists or a pharmaceutically acceptable salt, prodrug and/or solvate thereof, in combination with the HTS treatment, or ITS treatment to a subject in need thereof.

The present application also includes a use of one or more neuronal agonists, or a pharmaceutically acceptable salt, prodrug and/or solvate thereof, for improving the efficacy of HTS treatment or ITS treatment, as well as a use of one or more neuronal agonists, or a pharmaceutically acceptable salt, prodrug and/or solvate thereof, for the preparation of a medicament for improving the efficacy of HTS treatment or ITS treatment. The application further includes one or more neuronal agonists or a pharmaceutically acceptable salt, prodrug and/or solvate thereof, for use in improving the efficacy of HTS treatment or ITS treatment.

In an embodiment, improving the efficacy of HTS treatment or ITS treatment is by increasing the duration and/or or intensity of the HTS-mediated treatment effects or ITS-mediated treatment effects. In an embodiment, the duration of HTS- or ITS-mediated treatment effects are increased by about 10 to about 45 minutes, by about 20 minutes to about 40 minutes or by about 25 minutes to by about 35 minutes. In an embodiment, the duration of HTS- or ITS-mediated treatment effects are increased by about 30 minutes.

In an embodiment, the duration of HTS-mediated treatment effects are increased by about 10 to about 45 minutes, by about 20 minutes to about 40 minutes or by about 25 minutes to by about 35 minutes in a subject with cystic fibrosis. In an embodiment, the duration of HTS-mediated treatment effects are increased by about 30 minutes in a subject with cystic fibrosis.

In an embodiment, improving the efficacy of HTS treatment is by increasing the duration or intensity of HTS- or ITS-triggered mucociliary clearance. In an embodiment, the duration or intensity of the HTS- or ITS-triggered mucociliary clearance is increased by about 10 to about 90 minutes, by about 20 minutes to about 75 minutes, by about 30 minutes to about 75 minutes, by about 40 minutes to about 75 minutes, by about 45 minutes to by about 70 minutes, by about 50 minutes to by about 70 minutes or about 60 minutes. In an embodiment, the duration or intensity of the HTS- or ITS-triggered mucociliary clearance is increased by about 40 minutes to about 75 minutes, by about 45 minutes to by about 70 minutes, by about 50 minutes to by about 70 minutes or about 60 minutes. In an embodiment, the increasing the duration or intensity of the HTS- or ITS-triggered mucociliary clearance is in a subject with a non-cystic fibrosis respiratory disease, disorder or condition. In an embodiment, improving the efficacy of HTS treatment is by increasing the duration or intensity of HTS-triggered mucociliary clearance. In an embodiment, improving the efficacy of HTS treatment is by increasing the duration or intensity of HTS-triggered mucociliary clearance in a subject with a non-cystic fibrosis respiratory disease, disorder or condition.

In an embodiment, the one or more neuronal agonists or a pharmaceutically acceptable salt, prodrug and/or solvate thereof, are administered or used in combination with HTS treatment for improving the efficacy of HTS treatment.

In an embodiment, the one or more neuronal agonists or a pharmaceutically acceptable salt, prodrug and/or solvate thereof, are administered or used in combination with ITS treatment for in improving the efficacy of ITS treatment.

In an embodiment, the one or more neuronal agonists in combination with the HTS treatment or the ITS treatment are for administration or use in a subject with cystic fibrosis and/or a subject with one or more non-cystic fibrosis respiratory diseases, disorders or conditions. Accordingly, in an embodiment, the subject is a subject with cystic fibrosis and/or one or more non-cystic fibrosis respiratory diseases, disorders or conditions. In an embodiment, the subject is a subject with cystic fibrosis. In an embodiment, the subject is a subject with one or more non-cystic fibrosis respiratory diseases, disorders or conditions.

In an embodiment, the one or more non-cystic fibrosis respiratory diseases, disorders or conditions are selected from one or more of non-cystic fibrosis bronchiectasis, pneumonia, obstructive pulmonary disease (COPD), asthma, bronchiolitis, bronchitis, mucoid impaction (also known as mucus plugging) and primary ciliary dyskinesia. In an embodiment, the bronchiolitis is acute bronchiolitis. In an embodiment, the bronchiolitis is viral bronchiolitis. In an embodiment, the bronchitis is cigarette smoke-induced chronic bronchitis. In an embodiment, the respiratory disease, disorder or condition is non-cystic fibrosis bronchiectasis.

In an embodiment, the one or more non-cystic fibrosis respiratory diseases, disorders or conditions is a disease, disorder or condition characterized by an accumulation of and/or a difficulty in clearing of airway secretions, not including cystic fibrosis, arising from a neuromuscular disease, disorder or condition. In an embodiment, the neuromuscular disease, disorder or condition is selected from amyotrophic lateral sclerosis (ALS), a myopathy, a muscular dystrophy, and a spinal cord injury, and combinations thereof. In an embodiment, the muscular dystrophy is Duchenne muscular dystrophy. In an embodiment, the myopathy is a respiratory muscle weakness. In an embodiment, the one or more non-cystic fibrosis respiratory diseases, disorders or conditions is one or more diseases, disorders or conditions characterized by an accumulation of and/or a difficulty in clearing of airway secretions, not including cystic fibrosis, arising from rhinosinusitis. In an embodiment, rhinosinusitis is chronic rhinosinusitis.

In an embodiment, in the method of increasing the volume of an airway surface liquid (ASL) layer or treating a disease, disorder or condition that is treatable by increasing the volume of ASL layer, or enhancing hydration of an airway surface or treating a disease, disorder or condition that is treatable by enhancing hydration of an airway surface comprising administering an effective amount of one or more neuronal agonists or a pharmaceutically acceptable salt, prodrug and/or solvate thereof, in combination with hypertonic saline (HTS) treatment or isotonic saline (ITS) treatment, the subject is a subject with cystic fibrosis and/or one or more non-cystic fibrosis respiratory diseases, disorders or conditions.

In an embodiment, in the method of improving the efficacy of HTS treatment or ITS treatment comprising administering an effective amount of one or more neuronal agonists or a pharmaceutically acceptable salt, prodrug and/or solvate thereof, in combination with the HTS treatment or ITS treatment, the subject is a subject with cystic fibrosis and/or one or more non-cystic fibrosis respiratory diseases, disorders or conditions.

In an embodiment, in the method of increasing airway clearance or treating a disease, disorder or condition that is treatable by increasing airway clearance, or the method of increasing mucociliary clearance or treating a disease, disorder or condition that is treatable by increasing mucociliary clearance comprising administering an effective amount of one or more neuronal agonists or a pharmaceutically acceptable salt, prodrug and/or solvate thereof, in combination with the HTS treatment or ITS treatment, the subject is a subject with one or more non-cystic fibrosis respiratory diseases, disorders or conditions.

It would be appreciated by a person skilled in the art that a subject with cystic fibrosis can also have one or more non-cystic fibrosis respiratory diseases, disorders or conditions.

In an embodiment, the HTS treatment is for increasing the volume of an ASL layer or increasing mucociliary clearance or for treating a disease, disorder or condition treatable by increasing the volume of an ASL layer or increasing mucociliary clearance.

In the context of the present application, “increasing the volume of an ASL layer”, “enhancing hydration of an airway surface”, or “improving mucociliary clearance” refers to any increase in the volume of the ASL layer, any increase in hydration of an airway surface, or any increase in mucociliary clearance, respectively, when an effective amount of one or more neuronal agonists is used in combination with HTS treatment or ITS treatment compared to the volume of the ASL layer, the hydration of an airway surface or mucociliary clearance when HTS treatment or ITS treatment is used alone.

In the context of the present application, “improving the efficacy of HTS treatment or ITS treatment” refers to any increase in beneficial effects from HTS treatment or ITS treatment, respectively, for example, any increase in the volume of the ASL layer, any increase in hydration of an airway surface, or any increase in mucociliary clearance, when one or more neuronal agonists are used in combination with the HTS treatment or ITS treatment compared to when HTS treatment or ITS treatment is used alone.

In an embodiment the disease, disorder or condition that is treatable by increasing the volume of an ASL layer, and/or enhancing the hydration of an airway surface and/or increasing airway clearance and/or improving the efficacy HTS treatment or ITS treatment, is any disease, disorder or condition treatable using HTS treatment or ITS treatment.

In an embodiment, the disease, disorder or condition treatable using HTS treatment or ITS treatment is selected from cystic fibrosis and one or more non-cystic fibrosis respiratory diseases, disorders or conditions, and combinations thereof.

In an embodiment, the disease, disorder or condition treatable using HTS treatment or ITS treatment is cystic fibrosis.

In an embodiment, the disease, disorder or condition treatable using HTS treatment or ITS treatment is one or more non-cystic fibrosis respiratory diseases, disorders or conditions. In an embodiment, the one or more non-cystic fibrosis respiratory diseases, disorders or conditions are selected from one or more of non-cystic fibrosis bronchiectasis, pneumonia, obstructive pulmonary disease (COPD), asthma, bronchiolitis, bronchitis, mucoid impaction (also known as mucus plugging) and primary ciliary dyskinesia. In an embodiment, the bronchiolitis is acute bronchiolitis. In an embodiment, the bronchiolitis is viral bronchiolitis. In an embodiment, the bronchitis is cigarette smoke-induced chronic bronchitis. In an embodiment, the respiratory disease, disorder or condition is non-cystic fibrosis bronchiectasis.

In an embodiment, the one or more non-cystic fibrosis respiratory diseases, disorders or conditions is a disease, disorder or condition characterized by an accumulation of and/or a difficulty in clearing of airway secretions, not including cystic fibrosis, arising from a neuromuscular disease, disorder or condition. In an embodiment, the neuromuscular disease, disorder or condition is selected from amyotrophic lateral sclerosis (ALS), a myopathy, a muscular dystrophy, and a spinal cord injury, and combinations thereof. In an embodiment, the muscular dystrophy is Duchenne muscular dystrophy. In an embodiment, the myopathy is a respiratory muscle weakness. In an embodiment, the one or more non-cystic fibrosis respiratory diseases, disorders or conditions is one or more diseases, disorders or conditions characterized by an accumulation of and/or a difficulty in clearing of airway secretions, not including cystic fibrosis, arising from rhinosinusitis. In an embodiment, rhinosinusitis is chronic rhinosinusitis.

In an embodiment, the disease, disorder or condition treatable using HTS treatment or ITS treatment is cystic fibrosis and one or more non-cystic fibrosis respiratory diseases, disorders or conditions.

In an embodiment, the one or more neuronal agonists are selected from capsaicinoids, capsinoids, capsaisicin derivatives, monoterpenols, and combinations thereof. In an embodiment, the one or more neuronal agonists are selected from capsaicinoids, capsinoids, and monoterpenols, and combinations thereof. In an embodiment, the capsaicinoids are selected from capsaicin, dihydrocapsaicin, nordihydrocapsaicin, homocapsaicin, homodihydrocapsaicin and noivamide and combinations thereof. In an embodiment, the capsinoids are selected from capsiate, dihydrocapsiate and nordihydrocapsiate and combinations thereof. In an embodiment, the capsaisicin derivatives are selected from arvanil and olvanil and combinations thereof. In an embodiment, the monoterpenols are selected from monocyclic monoterpenols, bicyclic monoterpenols and monoterpenoid phenols. In an embodiment, the monocyclic monoterpenol is selected from menthol, isomenthol, carveol, terpineols, pulegol, isopulolegol, and hinokitiol and combinations thereof. In an embodiment, the bicyclic monoterpenols are selected from borneol, isoborneol, myrtenol and verbenol and combinations thereof. In an embodiment, the monoterpenoid phenols are selected from thymol, carvacrol and cumin alcohol and combinations thereof. In an embodiment, the monoterpenols are selected from monocyclic monoterpenols and bicyclic monoterpenols. In an embodiment, the monoterpenols are moncyclic monoterpenols. Therefore, in an embodiment, the one or more neuronal agonists are selected from capsaicinoids, capsinoids, capsaisicin derivatives, cyclic monoterpenols, bicyclic monoterpenols, and monoterpenoid phenols and combinations thereof. In an embodiment, the one or more neuronal agonists are selected from capsaicinoids, capsinoids, monocyclic monoterpenols and bicyclic monoterpenols, and combinations thereof. In an embodiment, the one or more neuronal agonists are selected from capsaicinoids, capsinoids, and monocyclic monoterpenols, and combinations thereof.

Accordingly, in an embodiment, the one or more neuronal agonists are selected from one or more of capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol isomenthol, carveol, terpineols, pulegol, isopulolegol, hinokitiol, borneol, isoborneol, myrtenol, verbenol, thymol, carvacrol and cumin alcohol. In an embodiment, the one or more neuronal agonists are selected from one or more of capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol, isomenthol, carveol, terpineols, pulegol, isopulolegol, hinokitiol, myrtenol, and verbenol. In an embodiment, the one or more neuronal agonists are selected from one or more of capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol, isomenthol, carveol, terpineols, pulegol, isopulolegol and hinokitiol. In an embodiment, the one or more neuronal agonists are selected from one or more of capsaicin, dihydrocapsaicin, nordihydrocapsaicin, capsiate, dihydrocapsiate, nordihydrocapsiate, arvanil, olvanil, menthol, isomenthol and isopulolegol. In an embodiment, the one or more neuronal agonists are selected from one or more of capsaicin, arvanil, olvanil, menthol, isomenthol, and isopulolegol. In an embodiment, the one or more neuronal agonists are selected from one or more of capsaicin, arvanil, olvanil, menthol and isopulolegol. In an embodiment, the one or more neuronal agonists are selected from one or more of capsaicin, menthol and isopulolegol. In an embodiment, the one or more neuronal agonists is selected from menthol and capsaicin, and a combination thereof. In an embodiment, the one or more neuronal agonists is menthol. In an embodiment, the one or more neuronal agonists is capsaicin. In an embodiment, the one or more neuronal agonists is menthol and capsaicin.

In an embodiment, the menthol is (−)-menthol, having the IUPAC name: (1R,2S,5R)-2-Isopropyl-5-methylcyclohexanol, and having the chemical formula:

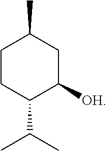

In an embodiment, the isopulegol is (−) isopulegol having the IUPAC name: (1R,2S,5R)-2-Isopropenyl-5-methylcyclohexanol, and having the chemical formula:

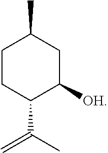

In an embodiment, the one or more neuronal agonists are transient receptor potential vanilloid type 1 (TRPV1) agonists and/or transient receptor potential cation channel subfamily M (melastatin) member 8 (TRPM8) agonists. In an embodiment, the one or more neuronal agonists are TRPV1 receptor agonists. In an embodiment, the one or more TRPV1 receptor agonists are selected from one or more of capsaicinoids, capsinoids, and capsaisicin derivatives. In an embodiment, the one or more TRPV1 receptor agonists is capsaicin. In an embodiment, the one or more neuronal agonists are one or more TRPM8 agonists. In an embodiment, the one or more TRPM8 agonists are selected from one or more of monoterpene alcohols (monoterpenols). In an embodiment, the one or more TRPM8 agonist is menthol. In an embodiment, the one or more neuronal agonists are one or more TRPM8 agonists and TRPV1 receptor agonists. In an embodiment, the TRPM8 agonist and TRPV1 receptor agonist is selected from one or more of monoterpenols. In an embodiment, the TRPM8 agonist and TRPV1 receptor agonist is isopulegol.

In an embodiment, the HTS treatment or the ITS treatment is administered as an aerosol. In an embodiment, the HTS treatment or the ITS treatment is nebulized.

In an embodiment, the subject is a mammal. In another embodiment, the subject is human.

In an embodiment, the HTS treatment or the ITS treatment is administered using an inhalation device. In an embodiment, the inhalation device is a nebulizer. In an embodiment, the nebulizer is a jet nebulizer, vibrating mesh (membrane) nebulizer or an ultrasonic nebulizer

In an embodiment, the hypertonic saline has a NaCl concentration of about 1% w/v to 40% w/v, about 1% w/v to about 35% w/v, about 1% w/v to about 30% w/v, about 1% w/v to about 25% w/v, about 1% w/v to about 20% w/v, about 1% w/v to about 15% w/v, about 1% w/v to about 10% w/v, about 2% w/v to about 8% w/v or about 3% w/v to about 7% w/v. In an embodiment, the HTS has a NaCl concentration of about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11 w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, or about 40% w/v. In an embodiment, the HTS has a NaCl concentration of about 1% w/v, about 2% w/v, about 3% w/v, about 4% w/v, about 5% w/v, about 6% w/v, about 7% w/v, about 8% w/v, about 9% w/v, or about 10% w/v. In an embodiment, the HTS has a NaCl concentration of about 3% w/v. In an embodiment, the HTS has a NaCl concentration of about 5% w/v. In an embodiment, HTS has a NaCl concentration of about 6% w/v. In an embodiment, the HTS has a NaCl concentration of about 7% w/v.

It will be appreciated that the effective amounts of the one or more neuronal agonists, or a pharmaceutically acceptable salt, prodrug and/or solvate thereof, and effective amounts of HTS or ITS will vary according to factors such as the disease state, age, sex and/or weight of the subject. In a further embodiment, the amount of a given neuronal agonists or neuronal agonists, or HTS or ITS, that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In an embodiment, the effective amount of the one or more neuronal agonists, or a pharmaceutically acceptable salt, prodrug and/or solvate thereof, is about 0.1 μM to about 1000 mM, about 0.1 μM to about 100 mM, about 0.5 μM to about 50 mM, or about 1 μM to about 10 mM.

It will also be appreciated that the effective amount of the one or more neuronal agonists, or a pharmaceutically acceptable salt, prodrug and/or solvate thereof, and effective amounts of HTS or ITS for administration or use may increase or decrease over the course of a particular regime. In some instances, chronic administration or use is required. In an embodiment, the one or more neuronal agonists, or a pharmaceutically acceptable salt, prodrug and/or solvate thereof, and HTS or ITS are administered or used in an amount and for a duration sufficient to control a disease, disorder or condition, or eliminate the disease, disorder or condition, treatable by HTS treatment or ITS treatment. In an embodiment, the one or more neuronal agonists, or a pharmaceutically acceptable salt, prodrug and/or solvate thereof, and HTS or ITS are administered or used in an amount and for a duration sufficient to control disease, disorder or condition treatable by HTS treatment or ITS treatment.

In an embodiment, the one or more neuronal agonists, or a pharmaceutically acceptable salt, prodrug and/or solvate thereof, and HTS or ITS are administered at least once a week. However, in another embodiment, the one or more neuronal agonists, or a pharmaceutically acceptable salt, prodrug and/or solvate thereof, and HTS or ITS are administered to the subject from about one time per two weeks, three weeks or one month. In another embodiment, the one or more neuronal agonists, or a pharmaceutically acceptable salt, prodrug and/or solvate thereof, and HTS or ITS are administered about one time per week to about once daily. In another embodiment, the one or more neuronal agonists, or a pharmaceutically acceptable salt, prodrug and/or solvate thereof, and HTS or ITS are administered 2, 3, 4, 5 or 6 times daily. The length of the treatment period depends on a variety of factors, such as the severity of the disease, disorder or condition, the age of the subject, the concentration and/or the activity the one or more neuronal agonists or a pharmaceutically acceptable salt, prodrug and/or solvate thereof, and HTS or ITS and/or a combination thereof. It will also be appreciated that the effective dosage of the one or more neuronal agonists, or a pharmaceutically acceptable salt, prodrug and/or solvate thereof, and the HTS or ITS used for the treatment may increase or decrease over the course of a particular treatment regime. Changes in dosage may result and become apparent by standard diagnostic assays known in the art. In some instances, chronic administration is required. For example, the one or more neuronal agonists, or a pharmaceutically acceptable salt, prodrug and/or solvate thereof, and HTS or ITS are administered to the subject in an amount and for duration sufficient to treat the subject.

The dosage of one or more neuronal agonists, or a pharmaceutically acceptable salt, prodrug and/or solvate thereof, and HTS or ITS varies depending on many factors such as the pharmacodynamic properties of the compounds, the mode of administration, the age, health and weight of the recipient, the nature and extent of the symptoms, the frequency of the treatment and the type of concurrent treatment, if any, and the clearance rate of the compounds in the subject to be treated. One of skill in the art can determine the appropriate dosage based on the above factors.

It is an embodiment that the one or more neuronal agonists, or a pharmaceutically acceptable salt, prodrug and/or solvate thereof, and HTS treatment or ITS treatment are administered contemporaneously. As used herein, “contemporaneous administration” of two substances to a subject means providing each of the two substances so that they are both active in the individual at the same time. The exact details of the administration will depend on the pharmacokinetics of the substances in the presence of each other, and can include administering the one or more neuronal agonists, or a pharmaceutically acceptable salt, prodrug and/or solvate thereof, and HTS or ITS within a few hours of each other, or even administering one substance within 24 hours of administration of the other, if the pharmacokinetics are suitable. Design of suitable dosing regimens is routine for one skilled in the art. In particular embodiments, the substances will be administered substantially simultaneously, i.e., within minutes of each other, or in a single composition that contains all substances. It is a further embodiment of the present application that a combination of agents is administered to a subject in a non-contemporaneous fashion. In an embodiment, the one or more neuronal agonists, or a pharmaceutically acceptable salt, prodrug and/or solvate thereof and HTS or ITS are administered simultaneously or sequentially in separate unit dosage forms or together in a single unit dosage form. In an embodiment, the one or more neuronal agonists, or a pharmaceutically acceptable salt, prodrug and/or solvate thereof and HTS or ITS are administered in a single unit dosage form.

It is an embodiment that the one or more neuronal agonists, or a pharmaceutically acceptable salt, prodrug and/or solvate thereof and HTS treatment or ITS treatment are administered or used according to treatment protocol that is known for administering HTS or ITS, respectively.

In an embodiment, the methods of the present application further comprise a pretreatment of the subject with an effective amount of a bronchodilator. In an embodiment, the bronchodilator is a beta-2 agonist. In an embodiment, the beta-2 agonists is selected from albuterol, salbutamol, and formoterol. In an embodiment, the bronchodilator is an anticholinergic. In an embodiment, the anticholinergic is ipratropium.

In an embodiment, the present application also includes a method of treating a disease, disorder or condition that is treatable by HTS treatment or ITS treatment comprising administering a therapeutically effective amount of one or more neuronal agonists, or a pharmaceutically acceptable salt, prodrug and/or solvate thereof, in combination with HTS treatment or ITS treatment, further in combination with another known agent useful for treatment of a disease, disorder or condition treatable by HTS treatment or ITS treatment. When used in combination with other agents useful for treating a disease, disorder or condition treatable by HTS treatment or ITS treatment, it is an embodiment that the one or more neuronal agonists, or a pharmaceutically acceptable salt, prodrug and/or solvate thereof, and HTS treatment or ITS treatment are administered contemporaneously with those agents. The exact details of the administration will depend on the pharmacokinetics of the substances in the presence of each other, and can include administering the one or more neuronal agonists, or a pharmaceutically acceptable salt, prodrug and/or solvate thereof, in combination HTS treatment or ITS treatment and the other agent(s) within a few hours of each other, or even administering one substance within 24 hours of administration of the others, if the pharmacokinetics are suitable. Design of suitable dosing regimens is routine for one skilled in the art. In particular embodiments, the substances will be administered substantially simultaneously, i.e., within minutes of each other, or in a single composition that contains all substances. It is a further embodiment of the present application that a combination of agents is administered to a subject in a non-contemporaneous fashion. In an embodiment, the one or more neuronal agonists, or a pharmaceutically acceptable salt, prodrug and/or solvate thereof, and HTS treatment or ITS treatment is administered with another therapeutic agent simultaneously or sequentially in separate unit dosage forms or together in a single unit dosage form.

In an embodiment, the another known agent useful for treatment of a disease, disorder or condition treatable by HTS treatment or ITS treatment is mannitol.

In an embodiment, the dosage of the one or more neuronal agonists, or a pharmaceutically acceptable salt, prodrug and/or solvate and the HTS or ITS treatment is equal to or less than the dosage of each of such agents when used alone. Such dosages are known to or readily determined by those skilled in the art.

In an embodiment, the subject is a mammal. In another embodiment, the subject is human.

The neuronal agonists of the application, for example, menthol, capsaicin, olvanil and arvanil, are available from commercial sources or can be prepared using methods known in the art. For example, the exemplary neuronal agonists of the application, menthol and/or capsaicin can be purchased from Sigma-Aldrich.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Examples of suitable solvate solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

### III. Compositions of the Application

The present application also includes a composition comprising one or more neuronal agonists, or a pharmaceutically acceptable salt, prodrug and/or solvate thereof, and HTS, wherein the one or more neuronal agonists, or a pharmaceutically acceptable salt, prodrug and/or solvate thereof, and HTS are present in amounts effective to increase the volume of an ASL layer and/or for treating a disease, disorder or condition treatable by increasing the volume of an ASL layer.

The present application also includes a composition comprising one or more neuronal agonists, or a pharmaceutically acceptable salt, prodrug and/or solvate thereof, and ITS, wherein the one or more neuronal agonists, or a pharmaceutically acceptable salt, prodrug and/or solvate thereof, and ITS are present in amounts effective to increase the volume of an ASL layer and/or for treating a disease, disorder or condition treatable by increasing the volume of an ASL layer.

The present application also includes a composition comprising one or more neuronal agonists, or a pharmaceutically acceptable salt, prodrug and/or solvate thereof, and HTS, wherein the one or more neuronal agonists, or a pharmaceutically acceptable salt, prodrug and/or solvate thereof, and HTS are present in amounts effective to enhance hydration of an airway surface or for treating a disease, disorder or condition treatable by enhancing hydration of an airway surface.

The present application also includes a composition comprising one or more neuronal agonists, or a pharmaceutically acceptable salt, prodrug and/or solvate thereof, and ITS, wherein the one or more neuronal agonists, or a pharmaceutically acceptable salt, prodrug and/or solvate thereof, and ITS are present in amounts effective to enhance hydration of an airway surface or for treating a disease, disorder or condition treatable by enhancing hydration of an airway surface.

The present application also includes a composition comprising one or more neuronal agonists, or a pharmaceutically acceptable salt, prodrug and/or solvate thereof, and HTS, wherein the one or more neuronal agonists, or a pharmaceutically acceptable salt, prodrug and/or solvate thereof, and HTS are present in amounts effective to increase mucociliary clearance or for treating a disease, disorder or condition treatable by increasing mucociliary clearance.

The present application also includes a composition comprising one or more neuronal agonists, or a pharmaceutically acceptable salt, prodrug and/or solvate thereof, and ITS, wherein the one or more neuronal agonists, or a pharmaceutically acceptable salt, prodrug and/or solvate thereof, and ITS are present in amounts effective to increase mucociliary clearance or for treating a disease, disorder or condition treatable by increasing mucociliary clearance

The present application also includes a composition comprising one or more neuronal agonists, or a pharmaceutically acceptable salt, prodrug and/or solvate thereof, and HTS, wherein the one or more neuronal agonists, or a pharmaceutically acceptable salt, prodrug and/or solvate thereof, and HTS are present in amounts effective to improve the efficacy of HTS treatment.

The present application also includes a composition comprising one or more neuronal agonists, or a pharmaceutically acceptable salt, prodrug and/or solvate thereof, and ITS, wherein the one or more neuronal agonists, or a pharmaceutically acceptable salt, prodrug and/or solvate thereof, and ITS are present in amounts effective to improve the efficacy of ITS treatment.

The one or more neuronal agonists, or a pharmaceutically acceptable salt, prodrug and/or solvate thereof, and HTS or ITS are suitably formulated into pharmaceutical compositions for administration to subjects in a biologically compatible form suitable for administration in vivo. Accordingly, the present application further includes a pharmaceutical composition comprising one or more neuronal agonists, or a pharmaceutically acceptable salt, prodrug and/or solvate thereof, and HTS and a pharmaceutically acceptable carrier. The present application further includes a pharmaceutical composition comprising one or more neuronal agonists, or a pharmaceutically acceptable salt, prodrug and/or solvate thereof, and ITS and a pharmaceutically acceptable carrier.

The present application also includes a kit for increasing the volume of an ASL layer or for treating a disease, disorder or condition treatable by increasing the volume of an ASL layer comprising one or more neuronal agonists, or a pharmaceutically acceptable salt, prodrug and/or solvate thereof, and HTS and optionally instructions for administration of the one or more neuronal agonists, or a pharmaceutically acceptable salt, prodrug and/or solvate thereof, in combination with the HTS.

The present application also includes a kit for increasing the volume of an ASL layer or for treating a disease, disorder or condition treatable by increasing the volume of an ASL layer comprising one or more neuronal agonists, or a pharmaceutically acceptable salt, prodrug and/or solvate thereof, and ITS and optionally instructions for administration of the one or more neuronal agonists, or a pharmaceutically acceptable salt, prodrug and/or solvate thereof, in combination with the ITS.

The present application also includes a kit for enhancing hydration of an airway surface or for treating a disease, disorder or condition treatable by enhancing hydration of an airway surface comprising one or more neuronal agonists, or a pharmaceutically acceptable salt, prodrug and/or solvate thereof, and HTS and optionally instructions for administration of the one or more neuronal agonists, or a pharmaceutically acceptable salt, prodrug and/or solvate thereof, in combination with the HTS.

The present application also includes a kit for enhancing hydration of an airway surface or for treating a disease, disorder or condition treatable by enhancing hydration of an airway surface comprising one or more neuronal agonists, or a pharmaceutically acceptable salt, prodrug and/or solvate thereof, and ITS and optionally instructions for administration of the one or more neuronal agonists, or a pharmaceutically acceptable salt, prodrug and/or solvate thereof, in combination with the ITS.

The present application also includes a kit for increasing airway clearance or for treating a disease, disorder or condition treatable by increasing airway clearance comprising one or more neuronal agonists, or a pharmaceutically acceptable salt, prodrug and/or solvate thereof, and HTS and optionally instructions for administration of the one or more neuronal agonists, or a pharmaceutically acceptable salt, prodrug and/or solvate thereof, in combination with the HTS.

The present application also includes a kit for increasing airway clearance or for treating a disease, disorder or condition treatable by increasing airway clearance comprising one or more neuronal agonists, or a pharmaceutically acceptable salt, prodrug and/or solvate thereof, and ITS and optionally instructions for administration of the one or more neuronal agonists, or a pharmaceutically acceptable salt, prodrug and/or solvate thereof, in combination with the ITS.

The present application also includes a kit for improving the efficacy of HTS treatment comprising one or more neuronal agonists, or a pharmaceutically acceptable salt, prodrug and/or solvate thereof, and HTS and optionally instructions for administration of the one or more neuronal agonists, or a pharmaceutically acceptable salt, prodrug and/or solvate thereof, in combination with the HTS.

The present application also includes a kit for improving the efficacy of ITS treatment comprising one or more neuronal agonists, or a pharmaceutically acceptable salt, prodrug and/or solvate thereof, and ITS and optionally instructions for administration of the one or more neuronal agonists, or a pharmaceutically acceptable salt, prodrug and/or solvate thereof, in combination with the ITS.

The one or more neuronal agonists, or a pharmaceutically acceptable salt, prodrug and/or solvate thereof, and HTS or ITS are administered to a subject in a variety of forms depending on the selected route of administration, as will be understood by those skilled in the art. For example, the one or more neuronal agonists, or a pharmaceutically acceptable salt, prodrug and/or solvate thereof, are administered by oral, inhalation, parenteral, buccal, sublingual, nasal, rectal, vaginal, patch, pump, topical or transdermal administration and the pharmaceutical compositions formulated accordingly. In an embodiment, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

In an embodiment, the one or more neuronal agonists or a pharmaceutically acceptable salt, prodrug and/or solvate thereof and the HTS or ITS in the compositions and kits of the present application are formulated as separate pharmaceutical compositions, which can be combined, for simultaneous administration to, or use in, subjects. In this embodiment, the separate pharmaceutical compositions are formulated independently of each other.

In an embodiment, the one or more neuronal agonists, or a pharmaceutically acceptable salt, prodrug and/or solvate thereof, and the HTS or ITS in the compositions and kits of the present application are formulated as a single pharmaceutical composition, for simultaneous administration to, or use in, subjects. In this embodiment, the one or more neuronal agonists, or a pharmaceutically acceptable salt, prodrug and/or solvate thereof, and the HTS or ITS are formulated together in a single unit dosage form.

In an embodiment, the one or more neuronal agonists, or a pharmaceutically acceptable salt, prodrug and/or solvate thereof are, formulated for administration, or use, by inhalation. In an embodiment, the HTS or ITS is formulated for administration, or use, by inhalation.

In an embodiment, the one or more neuronal agonists, or a pharmaceutically acceptable salt, prodrug and/or solvate thereof are, formulated for administration, or use, by sinonasal nebulization. In an embodiment, the HTS or ITS is formulated for administration, or use, by sinonasal nebulization. In an embodiment, the compositions are formulated administration by, or use with, a nebulizer.

In an embodiment, the HTS or ITS is contained in a vial or an ampoule. In an embodiment, the HTS or ITS is contained in a bottle. In an embodiment, the HTS or ITS is contained in an injection bag. In an embodiment, the HTS or ITS is contained in a cartridge.

In an embodiment, the vial is a single use vial or single use ampoule. In an embodiment, the vial or ampoule is filled with about 1 mL to about 10 mL, about 1 mL to about 8 mL, about 2 mL to about 7 mL, about 2 mL to about 5 mL, about 3 mL to about 5 mL or about 4 mL to about 5 mL of the HTS. In an embodiment, the vial or ampoule is filled with about 4 mL the hypertonic saline solution

In an embodiment, the kits comprise one or more of the vials or ampoules. In an embodiment, the kits of the application further comprise a nebulizer.

In an embodiment, the compositions are formulated for administration, or use, one to four times a day. In an embodiment, the compositions are formulated for administration, or use, two times a day. In an embodiment, the compositions of the application are formulated for administration, or use, once daily.

In an embodiment, the administration, or use, is carried out for a time about 1 minute up to about 30 minutes, for a time of about 10 minute up to about 30 minutes, for a time about 10 minutes up to about 20 minutes, for a time about 15 minutes up to about 20 minutes or for a time about 10 minutes up to about 15 minutes. In an embodiment, the administration, or use, is carried out for a time about 10 minutes up to about 15 minutes.

In an embodiment, the HTS or ITS comprises the one or more neuronal agonists, or a pharmaceutically acceptable salt, prodrug and/or solvate thereof.

In an embodiment, the hypertonic saline has an NaCl concentration of about 0.9% w/v.

In an embodiment, the hypertonic saline has an NaCl concentration of about 1% w/v to 40% w/v, about 1% w/v to about 35% w/v, about 1% w/v to about 30% w/v, about 1% w/v to about 25% w/v, about 1% w/v to about 20% w/v, about 1% w/v to about 15% w/v, about 1% w/v to about 10% w/v, about 2% w/v to about 8% w/v or about 3% w/v to about 7% w/v. In an embodiment, the HTS has an NaCl concentration of about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11 w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, or about 40% w/v. In an embodiment, the HTS has a NaCl concentration of about 1% w/v, about 2% w/v, about 3% w/v, about 4% w/v, about 5% w/v, about 6% w/v, about 7% w/v, about 8% w/v, about 9% w/v, or about 10% w/v. In an embodiment, the HTS has a NaCl concentration of about 3% w/v. In an embodiment, the HTS has a NaCl concentration of about 5% w/v. In an embodiment, HTS has a NaCl concentration of about 6% w/v. In an embodiment, the HTS has an NaCl concentration of about 7% w/v.

In an embodiment, the one or more neuronal agonists are selected from capsaicinoids, capsinoids, capsaisicin derivatives, menthol and menthol derivatives, and combinations thereof. In an embodiment, the one or more neuronal agonists are selected from capsaicinoids, capsinoids, menthol and menthol derivatives, and combinations thereof. In an embodiment, the capsaicinoids are selected from capsaicin, dihydrocapsaicin, nordihydrocapsaicin, homocapsaicin, homodihydrocapsaicin and noivamide. In an embodiment, the capsinoids are selected from capsiate, dihydrocapsiate and nordihydrocapsiate and combinations thereof. In an embodiment, the capsaisicin derivatives are selected from arvanil and olvanil and combinations thereof. Accordingly, in an embodiment, the one or more neuronal agonists are selected from capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol and menthol derivatives, and combinations thereof. In an embodiment, the one or more neuronal agonists are selected from capsaicin, dihydrocapsaicin, nordihydrocapsaicin, capsiate, dihydrocapsiate, nordihydrocapsiate, arvanil, olvanil, menthol and menthol derivatives, and combinations thereof. In an embodiment, the one or more neuronal agonists are selected from capsaicin, arvanil, olvanil, menthol and menthol derivatives, and combinations thereof. In an embodiment, the one or more neuronal agonists are selected from capsaicin, arvanil, olvanil and menthol, and combinations thereof. In an embodiment, the one or more neuronal agonists is selected from menthol and capsaicin, and a combination thereof. In an embodiment, the one or more neuronal agonists is menthol. In an embodiment, the one or more neuronal agonists is capsaicin. In an embodiment, the one or more neuronal agonists is menthol and capsaicin.

In an embodiment, the compositions and/or kits further comprise another therapeutic agent.

In an embodiment, the another therapeutic agent is mannitol.

For administration by inhalation, the compositions are conveniently delivered in the form of a solution, dry powder composition or suspension from a pump spray container that is squeezed or pumped by the subject or as an aerosol spray presentation from a pressurized container or a nebulizer. Aerosol compositions typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In an embodiment, the pressurized container or nebulizer contains a solution or suspension of the active compound. Capsules and cartridges (made, for example, from gelatin) for use in an inhaler or insufflator are, for example, formulated containing a powder mix of an active substance, such as the one or more neuronal agonists, or a pharmaceutically acceptable salt, prodrug and/or solvate thereof, and a suitable powder base such as lactose or starch. The aerosol dosage forms can also take the form of a pump-atomizer.

It is also possible to freeze-dry the one or more neuronal agonists, or a pharmaceutically acceptable salt, prodrug and/or solvate thereof, and use the lyophilizate obtained, for example, for the preparation of products for inhalation.

Depending on the mode of administration, the pharmaceutical composition will comprise from about 0.05 wt % w/v to about 99 wt % w/v or about 0.10 wt % w/v to about 70 wt % w/v of one or more neuronal agonists, or a pharmaceutically acceptable salt, prodrug and/or solvate thereof, and HTS or ITS, and from about 1 wt % w/v to about 99.95 wt % w/v or about 30 wt % w/v to about 99.90 wt % w/v of a pharmaceutically acceptable carrier, all percentages by weight being based on the total composition.

The following non-limiting examples are illustrative of the present application. As is apparent to those skilled in the art, many of the details of the examples may be changed while still practicing the methods, compositions and kits described herein.

### IV. EXAMPLES

### Example 1: Nebulized Hypertonic Saline Triggers Nervous System-Mediated Active Liquid Secretion in Cystic Fibrosis Swine Trachea⁴⁵

### Materials and Methods

### Animals

Female and male wild-type pigs were purchased from the Prairie Swine Centre, University of Saskatchewan. One-week old (˜3 kg) piglets were used for in vivo imaging; ˜5-weeks-old juvenile pigs (˜15 kg) were used for isolated trachea experiments. In addition, for ex vivo airway imaging, CFTR−/− tracheas were obtained from 6 newborn (6 to 12 h after birth), gut-corrected CFTR knockout pigs (CFTR−/−; TgFABP>pCFTR pigs), purchased from Exemplar Genetics (Exemplar Genetics, Sioux Center, Iowa, USA). The tracheas were dissected within 30 minutes of euthanasia, clamped at both ends to prevent liquid entering the lumen and placed in ice-cold Krebs-Ringer saline solution²³. For the bicarbonate-free Krebs-Ringer saline solution, 25 mM NaHCO₃ was replaced with 24 mM NaCl and 1 mM HEPES, pH 7.4, equilibrated in 0242. Both solutions had similar osmotic pressures of 270±0.7 and 269±0.5 (n=6).

Synchrotron-Based x-Ray Imaging Set-Up

A previously reported synchrotron-based imaging method to quantify ASL secretion in the airway was used^{23,24}. Experiments were performed using the BioMedical Imaging and Therapy-Bending Magnet (BMIT-BM) beamline 0561-1, at the Canadian Light Source (CLS), Saskatchewan, Canada. The experimental hutch is located 25.5 meters away from the storage ring. Phase contrast imaging (PCI) was performed using monochromatic 33.4 KeV (λ=0.37 nm) x-ray for live pig imaging and 20 keV (λ=0.062 nm) x-rays for isolated trachea imaging, selected using a standard double-crystal monochromator. The beam size was 100.0 mm (wide)×8.0 mm (vertical). The distance between the sample and the detector was 100 cm (for in vivo imaging) and 65 cm (for ex vivo isolated trachea imaging). Images were captured using a Fiber Optic Camera (C4742-56-12HR, Hamamatsu Photonics, San Jose, Calif., USA) or a high-resolution x-ray converter (AA-60, Hamamatsu Photonics, San Jose, Calif., USA) with a charge-coupled device (CCD) detector (C9300-124, Hamamatsu Photonics, San Jose, Calif., USA). The pixel size of the image was 11.2×11.2 μm (Fiber Optic Camera) or 8.75×8.75 μm (CCD camera). Exposure time ranged from 500 to 900 ms.

### Agarose Bead Preparation

Agarose beads were used as ‘measuring rods’ to determine the height of the ASL layer, which was measured as the distance between the air/ASL layer interface and the edge of the agarose bead touching the surface epithelia, as described in detail elsewhere (Luan, X. et al. Cystic fibrosis swine fail to secrete airway surface liquid in response to inhalation of pathogens. Nat. Commun. 8, 786, 10.1038/s41467-017-00835-7 (2017)²⁴. Agarose beads were made in sterile conditions with 4% w/v agarose in phosphate-buffered saline (PBS) as described^{23,24}. All solutions used in the process of making agarose beads were autoclaved at 250° C. for at least 20 min. Warm (50-55° C.) 4% w/v agarose solution was made in PBS. The agarose solution was mixed with warm (50-55° C.) heavy paraffin oil and stirred rapidly, achieving a vortex 2 cm in depth. The agarose/oil mixed solution was left in a beaker for 11 min, and then ice was slowly added around the beaker for 7 min. After 7 min of cooling, the agarose/oil solution was poured into a separatory funnel containing warm (50-55° C.) 0.5% w/v sodium deoxycholate in PBS to wash the mineral oil from the beads. The agarose beads were allowed to settle and then washed three times with PBS at room temperature to wash away the sodium deoxycholate. The upper one third of agarose beads at the bottom of the separatory funnel were then taken and used for the experiments. Agarose beads loaded with lidocaine, and capsaicin were prepared by mixing chemicals with warm (50-55° C.) 4% w/v agarose solution right before gelation procedure.

Preliminary experiments showed that beads are not visible unless a contrast agent was added. Thus, in order to make the beads visible by x-ray, BaSO₄ (nominal 1 M) as contrast agents was added to the PBS. This salt was chosen because it is insoluble in water and does not contribute to the osmotic pressure of the bead. The osmolarity of the beads solutions were 278±0.1 and 276±0.3 for PBS and PBS plus 1M BaSO4, respectively23.

### Experimental Set-Up

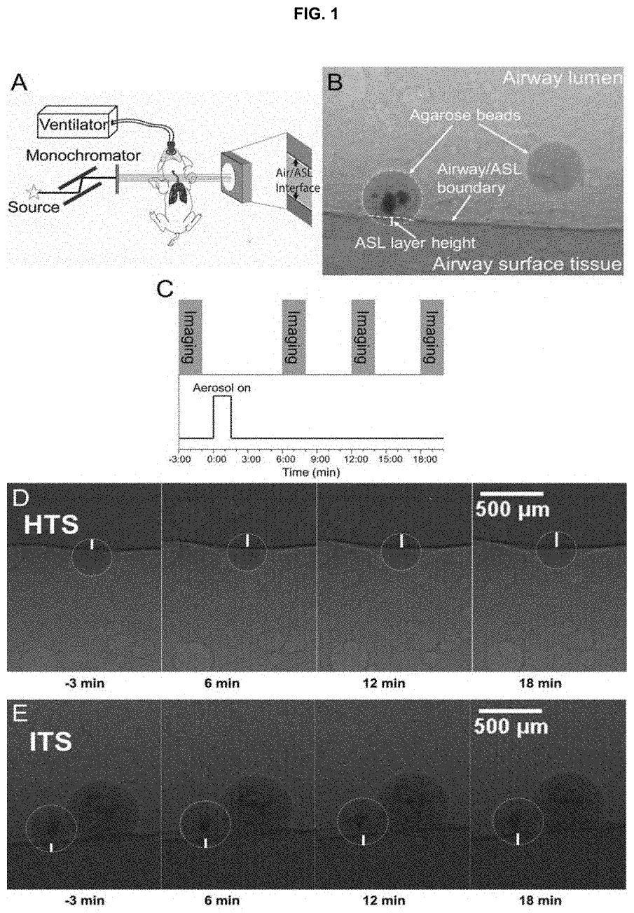

For in vivo swine imaging, the animal was held in supine position and fitted with a mask connected to an anesthetic machine providing 2% w/v isoflurane in pure medical oxygen at 1l/min (FIG. 1 A). Throughout the experiment, the animals breathed spontaneously. Respiratory rate, heart rate, body temperature, and O₂ saturation level as well as the plane of anesthesia were monitored. The larynx was sprayed with lidocaine to prevent a reflex response in preparation for intubation for every experiment. An endotracheal tube was placed at the opening of the larynx into the trachea and the agarose beads (˜400 to 1200 μm in diameter)²⁴ were blown through the endotracheal tube into the trachea with a puff of air, after which, the endotracheal tube was immediately removed.

For ex vivo airway imaging, a trachea was clamped at both ends to prevent contamination of the lumen with blood or other fluids. The cartilage was removed with a scalpel and a fine blunt-ended elevator to improve access of the drugs to the epithelia and nervous tissue. The trachea preparation was placed in a custom-built chamber. The tissue was immersed in Krebs solution plus 1 μM indomethacin at 35° C. and equilibrated with 95% w/v O2 and 5% w/v CO2. The lumen of the trachea preparation remained free of solution and sealed to keep the lumen from desiccating, yet it could be opened and accessed by researchers to introduce the agarose beads. Agarose beads (˜400 to 1000 μm in diameter) were blotted dry and placed in the lumen of the preparation using a cotton swab²³. To test the effect of airway neurons and epithelia on HTS treatment, atropine, tetrodotoxin, CFTRinh172, NK-1 blocker L-703606, bumetanide, and niflumic acid were dissolved in Krebs solution using in the custom-built chamber.

Each bead was placed at a location where the air/ASL layer interface was parallel to the x-rays penetrating the sample, i.e. the top or bottom of the preparation^{23,24}, using a motorized computer-controlled experimental stage to rotate the preparation (i.e. isolated preparation and live animal). Hypertonic (7% w/v NaCl solution w/v) and isotonic (0.9% w/v NaCl solution w/v) saline treatments were administered using a nebulizer (705-445, AMG Medical Inc, Montreal, Quebec, Canada), which produces liquid aerosols with a median diameter of 4 μm. Nebulization was performed for a period of 90 s with delivery of a total amount of 0.3 ml of liquid. For in vivo experiments, nebulized liquid was directly delivered into the face mask covering the swine. For ex vivo experiments, aerosol was delivered through one end of the isolated trachea with the other end opened to allow excess aerosol to flow out of the preparation. Hypertonic saline or isotonic saline treatment began at time 0, and the ASL layer volume increased was tested 18 min after treatment. Images were captured 3 minutes before treatment (−3 min), and 6, 12, and 18 minutes after treatment (FIG. 1 C).

### ASL Layer Height (Volume) Measurement

The large refractive index difference between the air and the ASL layer, helps to produce a strong signal at the air/ASL layer interface, using phase contrast imaging (PCI) (FIG. 1 B)^{23,24,25,43,44}. Because PCI cannot delineate the ASL layer/tissue interface, the position of the tissue with respect to the air/ASL layer interface was established using agarose beads as “measuring rods”. The agarose beads are placed in the ASL layer and come into direct contact with the surface epithelium due to the force generated by the surface tension of the ASL layer, as shown elsewhere²⁴. The surface tension of the ASL layer immobilizes the beads on the surface of the epithelium (i.e. beads are not cleared away by airway cilia), and the liquid secreted by the airway, which would normally be cleared from the airway due to mucociliary clearance, is retained around the static bead, thereby allowing the accumulation of the ASL layer produced by fluid secretion to be measured^{23,24}. In a small number of cases, 1 in 29 of the in vivo experiments and 3 in 70 of the ex vivo experiments, the airway displayed a change in diameter during the experiment. Since this could affect the measurements, those experiments were removed from the data set to eliminate any possible artefacts that may confound the interpretation of the data.

A researcher blinded to the experimental conditions measured the height of the ASL layer as the distance between the air/ASL layer interface and the edge of the agarose bead touching the surface epithelium (FIG. 1 B)^{23,24.} The data is presented as the difference in ASL layer height (Δ Liquid layer) at every time point and the initial measurement 3 min before nebulization (time −3 min).

### Statistics

To test the effect of each experimental condition, the ASL layer height produced by each preparation at 18 min were compared using ANOVA and Tukey's multiple comparisons tests in GraphPad Prism 5 (GraphPad Software Inc., San Diego, Calif., US), with p<0.05 considered significant. Data are presented as mean±S.E.M, where each individual agarose bead is a data point^{23,24}.

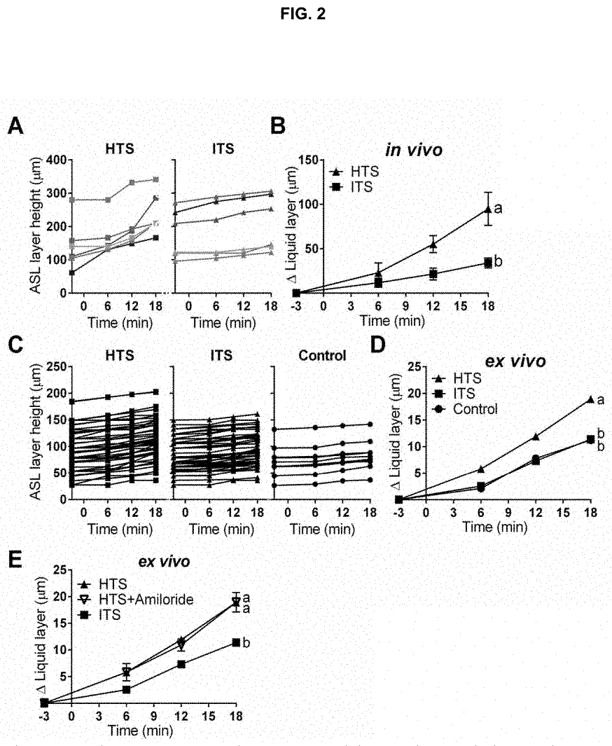

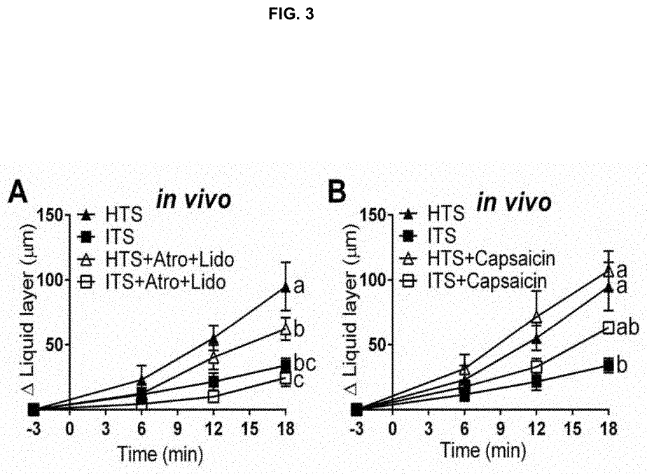

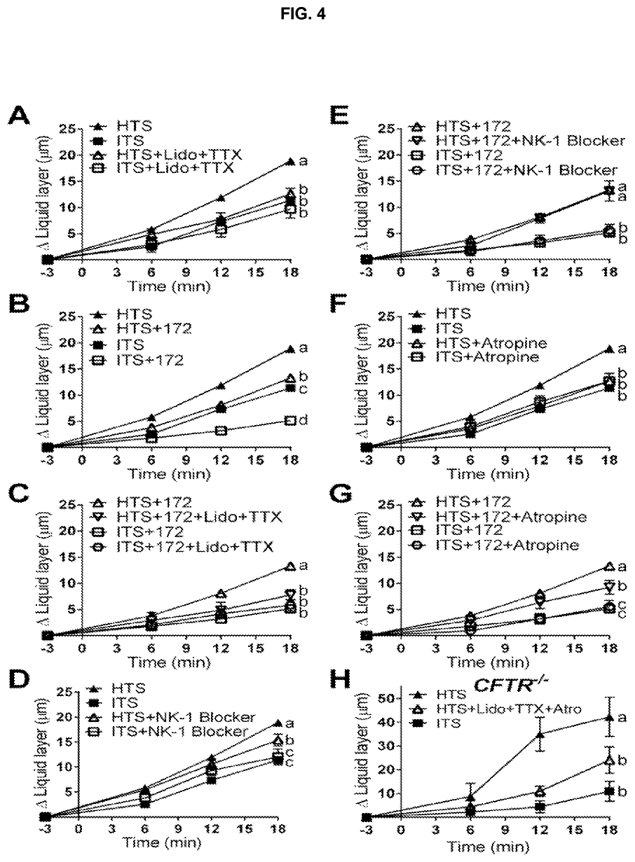

The data sets labeled HTS and (Isotonic Saline) ITS in FIG. 1 D are the same as those in FIGS. 2 A and B. Data sets labeled HTS and ITS in FIGS. 1 E and F are the same as those in FIGS. 3 A, B and D; and FIGS. 4 A and B. Data sets labeled HTS+172, and ITS+172 in FIG. 3 B are the same as those in FIGS. 3 C, E and G; and FIGS. 4 A, B and C.

### Reagents

Drugs were obtained from Sigma-Aldrich unless otherwise stated. CFTRinh172 was purchased from Cedarlane Labs (Burlington, ON, CA), tetrodotoxin was obtained from Alomone labs (Jerusalem, Israel), and lidocaine hydrochloride spray was acquired from Odan Laboratories LTD (Montreal, Canada). Stock solutions of CFTRinh172, lidocaine, atropine, bumetanide, niflumic acid, and L-703606 were dissolved in DMSO. The final concentration of DMSO was less than 0.1% w/v. Tetrodotoxin was directly dissolved into purified water.

### Study Approval

All experiments were performed with the approval of the Canadian Light Source and the Animal Ethics Committee at the University of Saskatchewan. All experiments were performed in accordance with relevant guidelines and regulations established by the Animal Ethics Committee at the University of Saskatchewan and the Canadian Council on Animal Care.

### Results

A synchrotron-based imaging method was used to quantify ASL secretion and determine the height of the ASL layer, as described elsewhere^{23,24} (FIG. 1, see methods). Nebulized hypertonic (7% w/v NaCl solution w/v) or isotonic (0.9% w/v NaCl solution w/v) saline was administered to live wild-type swine (FIG. 1 A)². Treating pigs (in vivo) with HTS significantly increased ASL layer height compared to preparations nebulized with isotonic saline (ITS) (FIGS. 2 A and B). A similar result was obtained from isolated tracheas (ex vivo), where HTS-treated preparations triggered greater ASL secretion than ITS-treated or untreated (control) ones (FIGS. 2 C and D). This suggests that HTS treatment does indeed increase ASL production in the preparations. To determine whether the ASL layer volume increase could be explained by the inhibition of ASL reabsorption by epithelial sodium channel (ENaC)-mediated pathway after HTS treatment²⁶, the ENaC inhibitor, amiloride, was added to the HTS treatment. The results showed that amiloride did not affect HTS treatment in the pig airway (FIG. 2 E), which indicates that HTS-triggered increase in ASL layer height results from the production of liquid into the airway lumen, and not by blocking liquid absorption.

### HTS-Triggered ASL Layer Height Increase is Partially Mediated by the Nervous System

Since previous reports indicated that HTS may stimulate sensory neurons and the autonomic nervous system^{13,18}, the effect of blocking the nervous system on HTS-triggered ASL layer height increase in vivo was tested. Agarose beads were used both as “measuring rods” (see methods section) and as vehicles to deliver lidocaine into the trachea. Agarose beads have been used as vehicles for drug delivery^{23,24} in previous studies; Luan et al showed that beads can be loaded with bacteria, LPS and flagellin and that these compounds leach out of the beads and stimulate submucosal gland ASL production in swine trachea^{23,24}.

Swine were treated with atropine (0.04 mg/kg, intramuscular)²⁷ to block the autonomic nervous system, and the agarose beads were loaded with lidocaine (80 mg/ml)²⁸ to inhibit sensory neurons in the airway. This treatment (HTS+Atro+Lido) reduced HTS-triggered (HTS) ASL layer height increase in vivo by ˜50% w/v. However, the atropine-plus-lidocaine treatment had no effect on ITS-treated swine (FIG. 3 A, supplementary table 1). These results suggest that HTS treatment, but not ITS, recruits the nervous system. Approximately 50% w/v of the HTS-triggered ASL production is mediated by stimulation of sensory neurons and autonomic nervous system.

Since HTS has been proposed to stimulate C-fibers²⁹, which also respond to capsaicin, whether capsaicin (10 μM) affected HTS treatment was tested³⁰. Agarose beads loaded with capsaicin (ITS+Cap) resulted in an increase in the ASL layer height greater than in swine treated with ITS (ITS) nebulization alone (FIG. 3 B) indicating that C-fiber stimulation triggers ASL production. Combining capsaicin with HTS (HTS+Cap) treatment showed slight improvement in HTS-triggered (HTS) ASL layer height (FIG. 3 B) at 18 minutes, suggesting that HTS and capsaicin may act on the same target (i.e. C-fibers). Suggesting that activation of airway neurons contributes to fluid secreted into the airway during HTS nebulization.

To further test this idea, isolated pig trachea preparations were used for more invasive experiments. It has been established that incubating trachea preparations in tetrodotoxin (TTX, 1 μM) and lidocaine (10 mg) (single luminal application of 80 mg/ml spray)²⁸ blocks both the voltage-dependent sodium channels expressed by neurons intrinsic to the airways^{31,32} and the TTX-independent sodium channels expressed by sensory neurons of the airways^{20,33} without directly altering epithelial ion secretion²⁴. It was found that blocking neuronal function with lidocaine+TTX reduced the effect of HTS treatment (FIG. 4 A). In contrast, lidocaine+TTX had no effect on ITS-treated preparations, suggesting that the effect of lidocaine+TTX is specific to HTS treatment (FIG. 4 A).

To evaluate the effect of HTS treatment in CF airways, the CFTR blocker CFTRinh172 (100 μM) was used in isolated wild-type trachea preparations to model airways without CFTR function³⁴. HTS treatment in preparations incubated with CFTRinh172 (HTS+172) was less effective in stimulating ASL layer height increase than preparations not incubated in CFTR blocker (HTS). However, preparations incubated with CFTRinh172 still produced greater ASL layer height increase when treated with HTS than ITS (FIG. 4 B). This finding is consistent with previous experiments showing that HTS treatment improves ASL layer hydration in CF patients^{35,38}. Interestingly, CFTRinh172 treatment reduced ASL secretion in ITS-treated samples (ITS+172) below that of ITS treatment in control preparations (ITS, FIG. 4 B), which, while not being limited by theory, may be explained by the blockage of CFTR-dependent basal ASL secretion in swine trachea as described in another study²⁴.

Preparations incubated with CFTRinh172 suffered a reduction in the response to HTS treatment when neuronal function was blocked with lidocaine+TTX (FIG. 4 C). In contrast, ITS-treated preparations were not affected by lidocaine+TTX (FIG. 4 C). These results suggest that HTS treatment, but not ITS, increases ASL layer height through stimulation of sensory and/or airway intrinsic neurons in CF airways.

Since there is evidence that HTS treatment triggers substance P (SP) release¹⁸ and that SP can stimulate ASL secretion by airway epithelia in the trachea of several species^{21,37,38}, the effect of a SP receptor (neurokinin 1 receptor, NK-1) blocker, L-703606 (1 μM) on HTS-stimulated ASL layer height increase was tested. Treatment with L-703606 (HTS+NK-1 Blocker) significantly reduced HTS-triggered ASL layer height increase, but had no effect on the ITS treatment group (ITS+NK-1 Blocker, FIG. 4 D). However, HTS treatment of preparations incubated with CFTRinh172 was unaffected by the NK-1 blocker (FIG. 4 E), indicating that the substance P-mediated HTS-triggered effect is CFTR-dependent and may be absent in CF airways.

The possible role of muscarinic stimulation on HTS-triggered ASL production was then tested. Blocking muscarinic receptors with atropine (1 μM)³⁸ reduced HTS-triggered ASL secretion but had no effect on ITS treated samples (FIG. 4 F). Atropine also reduced the response to HTS treatment in preparations incubated with the CFTR blocker CFTRinh172, but had no effect on ITS-treated preparations, suggesting a role for cholinergic signaling in HTS-triggered ASL secretion in both CF and non-CF airways.

To further test the contribution of the nervous system to HTS-triggered ASL secretion in CF, the effect of HTS on CFTR^{-/-} swine trachea preparations (FIG. 4 H) was studied. Treatment of CFTR^{-/-} tracheas with HTS significantly increased ASL layer height compared to ITS-treated preparations. The HTS effect was significantly reduced by treatment with blockers of the nervous system, lidocaine+TTX+atropine (FIG. 4 H). These results further corroborate that HTS treatment stimulates airway neurons which release acetylcholine on airway epithelia and trigger active ASL secretion in CF airways.

### HTS Treatment Triggers Active Secretion of ASL by Airway Epithelia

To investigate the possible role of active epithelial ASL secretion in response to HTS treatment, pharmacological agents that block epithelial ASL production in isolated trachea preparations were tested. If HTS triggers neuron-stimulated epithelial ASL secretion, the effect of HTS would be expected to be blocked by blocking ion transport by epithelial cells. However, if the effect of HTS is entirely mediated through osmosis, then an ion transport blocker treatment should not affect it. As most of the ASL in the upper airways (i.e. down to about the 10th bronchial generation, where airway lumen is ˜1-2 mm in diameter) is produced by submucosal glands³⁹, blockers known to abrogate gland secretions on HTS-triggered ASL production were tested: the CFTR blocker CFTRinh172, the Na⁺:K⁺:2Cl⁻ cotransporter blocker bumetanide (100 μM)³⁹, and a Ca²⁺-activated Cl⁻ channel blocker niflumic acid (100 μM)³⁶ in HCO₃⁻-free saline solution⁴⁰.

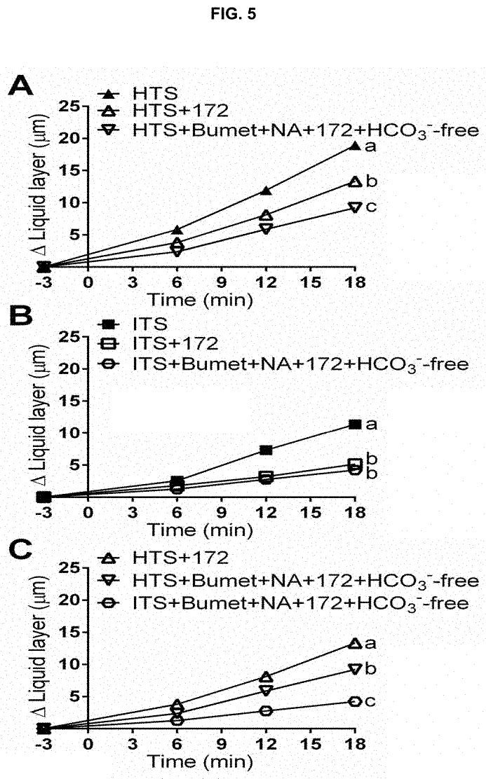

HTS treatment of both wild-type preparations incubated in the CFTR inhibitor CFTRinh172 as well as CFTR^{-/-} tracheas (FIG. 4 H), resulted in an increased in ASL layer height. This suggests that HTS may trigger ASL production by the airway epithelia in a CFTR-independent manner (FIG. 5 A). The CFTR-independent ion transport-related effect of HTS was blocked in wild-type preparations incubated in the cocktail of blockers (i.e. CFTRinh172, bumetanide, and niflumic acid in HCO₃⁻-free bath, FIG. 5 A). In contrast, ASL layer height in ITS-treated preparations was inhibited similarly by CFTR inhibitor alone and by the ion blocker cocktail (FIG. 5 B), indicating that the blocker cocktail specifically blocks the effect of HTS treatment. These results suggest that the CF airway epithelium responds to HTS treatment with active ASL secretion independent of CFTR.

To estimate the proportion of ASL produced by active ASL secretion by CF epithelia, preparations in CFTRinh172, bumetanide, and niflumic acid were incubated in HCO₃⁻-free saline and then treated with HTS or ITS (FIG. 5 C). Since these preparations could not produce ASL through epithelial secretion, the difference in ASL produced by HTS and ITS treated preparations should have been driven by the osmotic effect alone, generated by the hypertonic treatment. Incubation with HTS+Bumet+NA+172+HCO₃⁻ increased ASL layer height by 9.14±0.69 μm. ITS+Bumet+NA+172+HCO₃⁻ treatment produced an increase in ASL layer height of 4.23±0.5 μm. HTS+172 treatment increased ASL layer height by 13.32 μm. Since tissues incubated in Bumet+NA+172+HCO₃⁻ cannot produce active secretion of ASL, the difference between HTS treatment (9.14 μm) and ITS (4.23 μm) can only be the result of osmotic effect (due to the exclusion of active epithelial secretion). Thus, the ASL layer height produced by osmotic effect alone is 4.96 μm. The ASL produced by osmotic effect plus active secretion in a CF model is 9.09 μm, the difference between HTS+172 and ITS+Bumet+NA+172+HCO₃⁻. Hence, the osmotic effect alone, 4.96 μm, is 54% w/v of the osmotic plus active epithelial secretion, 9.09 μm. The results suggest that ˜50% w/v of the ASL produced by HTS treatment in CF is generated by active ASL secretion by airway epithelia.

### Discussion

It has been found that inhaled HTS treatment causes ASL production through the stimulation of the nervous system, which triggers active ASL secretion by airway epithelia. This pathway occurs alongside the osmotic effect of HTS that draws water from the serosal surface into the ASL layer¹². The presence of hypertonic saline may be detected by Aδ- and C-fibers^{16,17}, probably as a change in osmolality or ion concentration in the sensory neurons¹⁶. The local release of neurotransmitters may stimulate ASL secretion from airway submucosal glands³⁶, and possibly modulate surface airway epithelia ion transport⁴¹. Aδ- and C-fibers are more frequent in the larger airways¹⁹, which may explain the recent unexpected finding that HTS treatment has a stronger effect on mucociliary clearance in larger airways than in small ones¹⁰.

In the ex vivo preparations, substance P- and cholinergic-mediated stimulation of ASL secretion was detected. However, only cholinergic stimulation had an effect on preparations without functional CFTR, suggesting that the contribution of the nervous system on HTS-triggered ASL secretion is different in CF and non-CF airways²⁰. The present results indicate that it may be possible to modulate the duration and intensity of HTS treatment by pharmacologically modulating the contribution of the nervous system to HTS-triggered ASL secretion, which provides a potential target for drug development.

It has been also found that the use of HTS formulations which include blockers of epithelial ASL reabsorption (e.g. ENaC blockers) may reduce the effect of HTS treatment if they interfere with epithelial ASL secretion by altering the epithelium physiology or through non-specific effects on other ion transportersl. The involvement of active ASL secretion by the airway epithelia in HTS-triggered ASL production suggests the possibility that in addition to improving mucociliary clearance, hypertonic saline may also increase the production of epithelia-secreted molecules, such as mucin and antimicrobial compounds, that contribute to airway sterility.

### Example 2: Neuronal Agonist and Inhaled HTS or ITS

### Materials and Methods

### A. ASL Secretion

The material and methods used for the study of ASL secretion were as per Example 1. The menthol used for the study of ASL secretion and was (−)-menthol.

### B. Mucociliary Clearance

### Animals

The trachea was dissected from juvenile male and female swine and placed within an oxygenated ice-cold Krebs Ringer solution until used for experimentation. Clamps were applied at both ends of the trachea to prevent any fluid from entering the trachea. Experiments were performed typically within 4 hours of euthanasia on the same day.

### Dissection

A section of 1-1.5 inches was removed from the trachea. The lumen of the trachea preparation was rinsed in phosphate buffer solution (PBS) to remove debris or blood that may be present in the lumen. Subsequently, the cartilage rings were dissected. An incision was made across three cartilage rings; an elevator was used to separate the cartilage rings from the underlying mucosa. It was important that the mucosa remained intact, as fluid would enter the lumen of the trachea in later portions of the experiment otherwise. Using forceps and the elevator, the cartilage was pulled back laterally in both directions to expose a small section of the outer mucosa. This dissection typically took 4-8 minutes. At 6 minutes, Krebs solution was cautiously applied to the outside of the tissue to prevent it from drying out; it was important that no fluid entered the lumen of the tissue in the process. Once the dissection was completed, the tracheal preparation was clamped and bathed for 15 minutes in solutions containing the drugs to be tested.

### Inhaled HTS or ITS

To study the effect of inhaled isotonic saline (ITS) or hypertonic saline treatment, the lumen of the trachea preparation was connected with a nebulizer (MedPro, IN, USA). The tissue was nebulized for 90 seconds with either HTS or ITS. For the control group, no nebulization occurred.

### Computerized Imaging Set-Up and Placement of Particles

Immediately following nebulization, scissors were used to cut the tissue longitudinally at two locations to open the trachea. The trachealis smooth muscle was left intact, with an incision made on one side of it, and another incision made opposite to it. The tissue preparation was placed on a plate lined with gauze bandages that were soaked in Krebs solution containing the drug(s) to be tested. The purpose of using the gauze was to maintain humidity and tissue health during the experiment. Eight 250 μm diameter and 25 μm thickness tantalum disks were placed in two rows of four along the luminal surface at the distal portion of the trachea.

The tissue was then placed within a temperature- and oxygen-controlled chamber. The preparation was maintained at 37° C. and humidified with 95% w/vO₂/5% w/vCO₂. The position of the tantalum disks was recorded with a digital camera (MiniVid, GA, USA) every 30 seconds for as long as the disks were mobile.

### Analysis of Mucociliary Clearance (MCC) Rate

To monitor mucociliary clearance (MCC) rate, the image sequences were analyzed for numerous parameters. The primary measures of MCC included: maximum particle transport rate, average particle transport rate, distance travelled, number of mobile particles, and duration of particle mobility. Additional parameters included: the time that movement began and the time that maximum transport rate was reached.

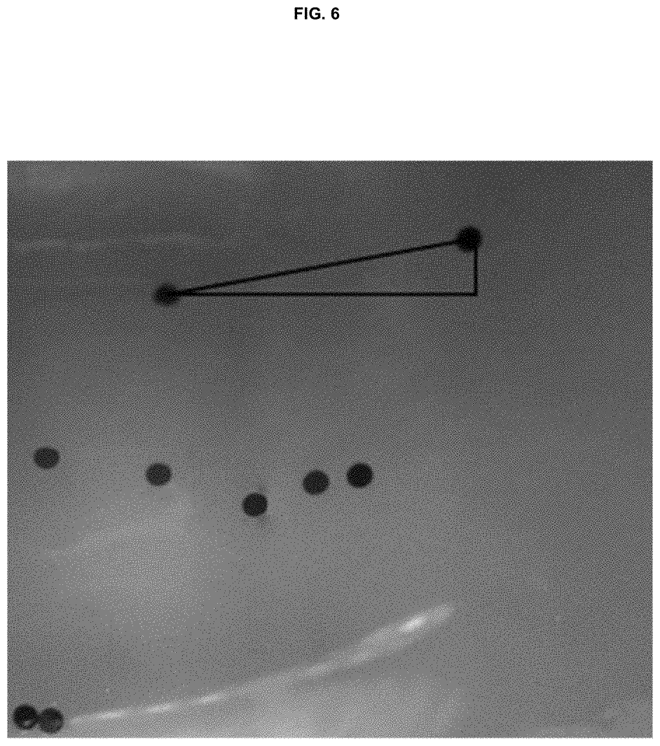

To quantify particle movement, the X and Y position of each particle was recorded in each image. The movement between consecutive images was measured using vectors of the differences in X and Y positions. These were used to calculate the hypotenuse of their movement (FIG. 6), which provided a measure of overall movement. Measurements may also be taken to calculate net movement toward the laryngeal end of the tissue. Results on distance travelled were then used to calculate speed.

### Exclusion/Inclusion Criteria

Not all experiments were included in the data analysis for this project. Experiments in which the mucosal layer was punctured were immediately disregarded, as fluid would enter the tracheal lumen and potentially influence particle movement. Furthermore, experiments with less than three mobile particles were excluded since it was a sign of tissue damage; preliminary experiments found that damaged tissues displayed movement of fewer particles. Damage to the luminal surface was unable to be observed directly, and damage could occur at a number of stages during the experimental protocol, including during dissection or cutting open the tissue. For this reason, it was decided that a reasonably healthy tissue would display movement of 3 or more particles, and this was ultimately used as the inclusion criteria.

### Statistical Analysis

To test the differences in MCC of each treatment group, the mean of each group's individual particles' maximum transport speed reached was compared using ANOVA and Tukey's multiple comparisons tests in GraphPad Prism 5 (GraphPad Software Inc., San Diego, Calif., US), with p<0.05 considered significant.

### Reagents

CFTRinh-172 was purchased from Cedarlane Labs (Burligton, ON, CA). Stock CFTRinh-172 solutions were dissolved in DMSO, with a final DMSO concentration of less than 1% w/v.

### Study Approval

All experiments were performed under the approval of the Animal Ethics Committee at the University of Saskatchewan. All of the guidelines and regulations established by the Animal Ethics Committee at the University of Saskatchewan and the Canadian Council on Animal Care were adhered to.

### Results and Discussion

### A. Time Course of HTS-Triggered ASL Secretion

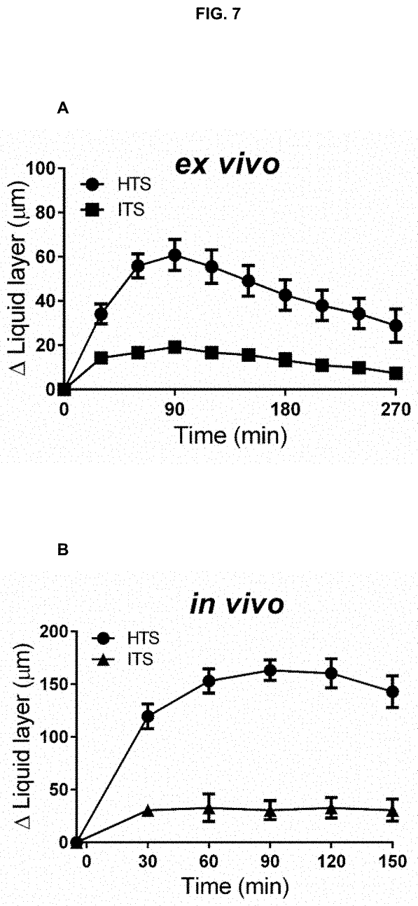

Using the synchrotron-based methodology a described in Example 1, the effect of treatment on the time course of HTS-triggered ASL layer volume was measured. It has been found that the increase in ASL layer volume induced by HTS peaks at ˜90 min after treatment in wild-type swine trachea preparations (ex vivo, FIG. 7 A) as well as in living swine (in vivo, FIG. 7 B).

It has been further found that HTS treatment produces less ASL secretion and its duration of peak fluid secretion is shortened by blocking the nervous system and active ion transport. These results further corroborate the involvement of the nervous system in mediating HTS' effects.

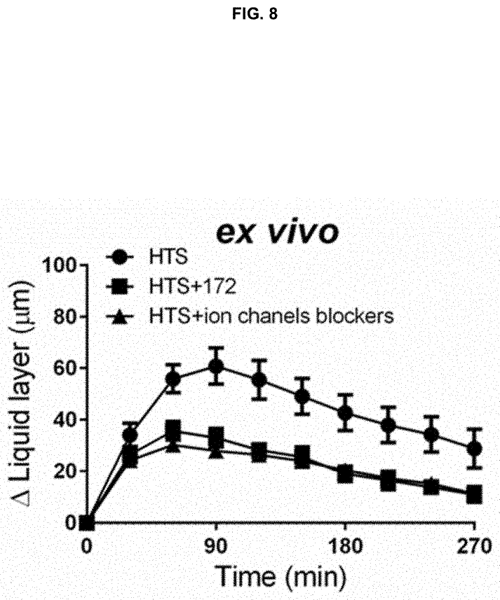

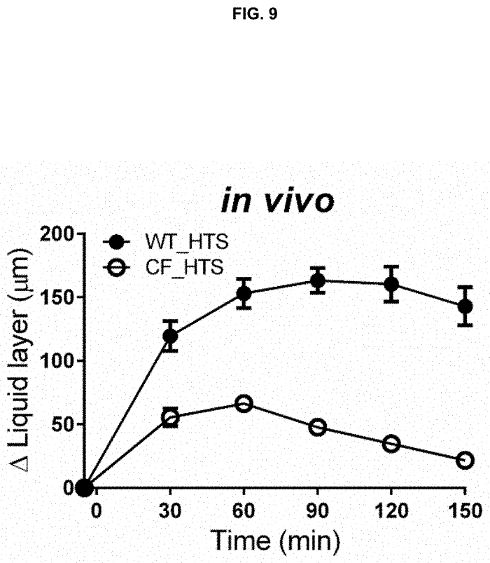

As shown in FIGS. 8 to 11, it has been surprisingly found that the time course of action of HTS can be adjusted through nervous system modulation. FIG. 8 shows that the use of ion transporter blockers (niflumic acid, bumetanide, CFTRinh172) and CFTR blocker CFTRinh172 (172) decreases the effect of HTS on ASL secretion in wild-type swine trachea preparations (ex vivo). Further, FIG. 9 shows that ASL secretion from HTS treatment peaks earlier in cystic fibrosis pigs (CFTR knockout) than wild-type. The cystic fibrosis pigs (CFTR) where purchased from Exemplar Genetics (Exemplar Genetics, Sioux Center, Iowa, USA). In addition, less fluid is produced. Thus, the effect of HTS on CF and non-CF airways is distinct.

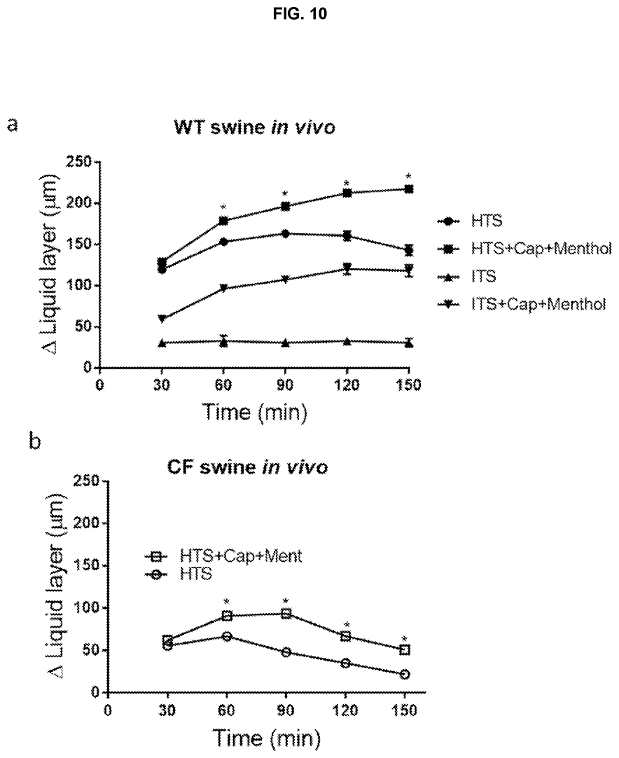

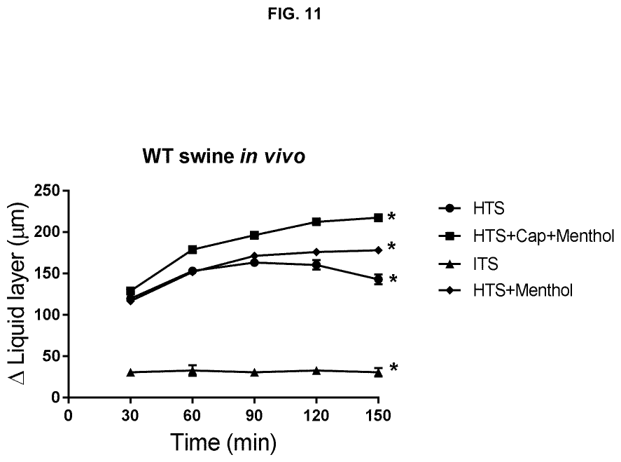

More surprisingly, it was unexpectedly found that adding exemplary neuronal agonists capsaicin (cap) and menthol (ment) to HTS or ITS increases the volume of fluid produced and prolongs the duration of action of HTS or ITS in wild-type swine in vivo (FIG. 10 A) and of HTS in CF swine (FIG. 10 B) in vivo. Similarly, adding exemplary neuronal agonist menthol alone to HTS was found to increase the intensity and duration of treatment in WT (wild type) swine in vivo but to a lesser extent than in conjunction with exemplary neuronal agonist capsaicin (FIG. 11).

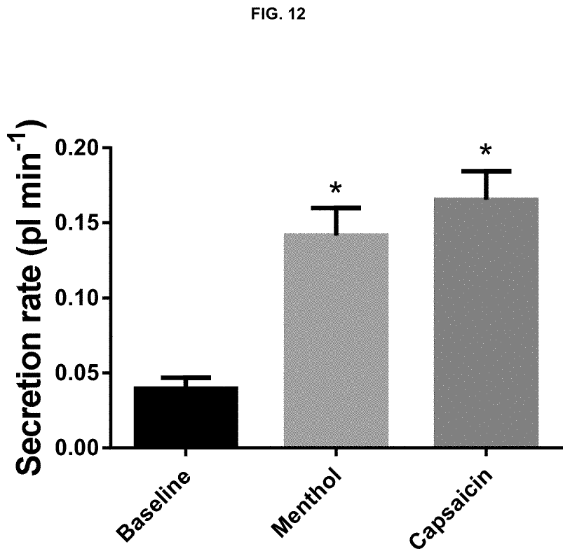

To confirm that both exemplary neuronal agonists capsaicin and menthol stimulate active fluid secretion by airway submucosal glands in WT swine, the effect of exemplary neuronal agonists menthol and capsaicin was on fluid secretion rate was measured using the secretion assay as described elsewhere²¹. The mucosal layer from trachea or primary bronchus, with underlying glands, was dissected from the cartilage. A piece of tissue of about 1 cm² was pinned mucosal side up and mounted in a 35-mm-diameter plastic Petri dish lined with Sylgard (Dow Corning Corp). The serosal side was immersed in a bathing solution (˜1 ml volume), and the mucosa was in air. Before experimentation, the mucosal surface was gently cleaned and blotted dry with cotton swabs and further dried with a stream of air, after water-saturated mineral oil was placed on the surface. The preparation was then placed in a temperature-controlled chamber (TC-324B; Warner Instruments, Hamden, Conn.) maintained at 37° C. and equilibrated with warmed, humidified 95% O₂-5% CO₂. Pharmacological agents were diluted to final concentrations with warmed bath solution equilibrated with 95% O₂-5% CO₂. They were added to the serosal side by complete bath replacement. Droplets of ASL form under oil during secretion by individual submucosal glands. Images of the lumen of the trachea were taken at 2-min intervals using a digital camera (MiniVid; LW Scientific, Lawrenceville, Ga.) and stored for offline analysis. Stored images were analyzed using ImageJ 1.32J (NIH). Secretion volumes were calculated, as previously described, by assuming the mucus droplet to be spherical. Then V=4/3π r3, where r is the radius. Exposing the mucosal side to menthol or capsaicin triggers significant ASL secretion by the submucosal glands as shown in FIG. 12.

### B. Mucociliary Clearance

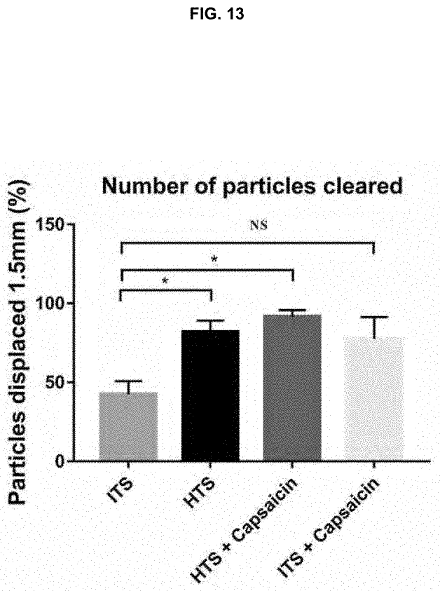

The mucociliary clearance assay described under materials and methods which assesses mucociliary clearance by measuring the movement of tantalum disks across a segment of excised swine trachea was used to assess the effect of HTS and ITS each alone and in combination with exemplary neuronal agonist capsaicin on mucociliary clearance. As shown in FIG. 13, in WT swine airways, HTS-triggered or ITS-triggered increase in mucociliary clearance is augmented by the addition of exemplary neuronal agonist capsaicin.

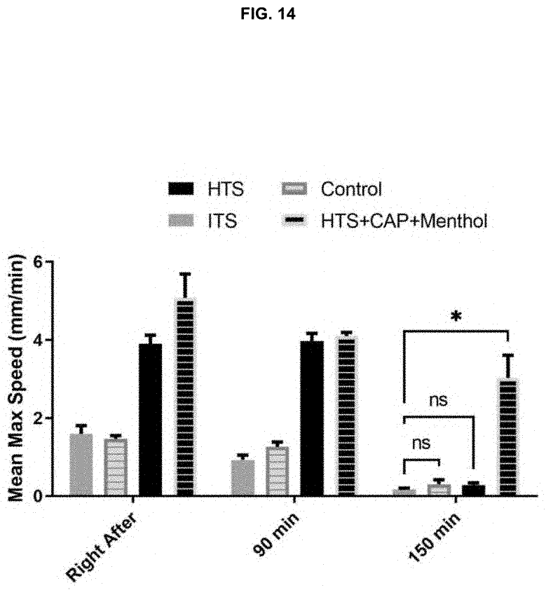

As further shown in FIG. 14, the addition of exemplary neuronal agonists menthol and capsaicin to HTS increased the duration of HTS-triggered mucociliary clearance by 60 min in wild-type swine. These results suggest that combining HTS with neuronal agonists may have beneficial therapeutic effects, for example, in non-cystic fibrosis respiratory diseases, disorders or conditions.

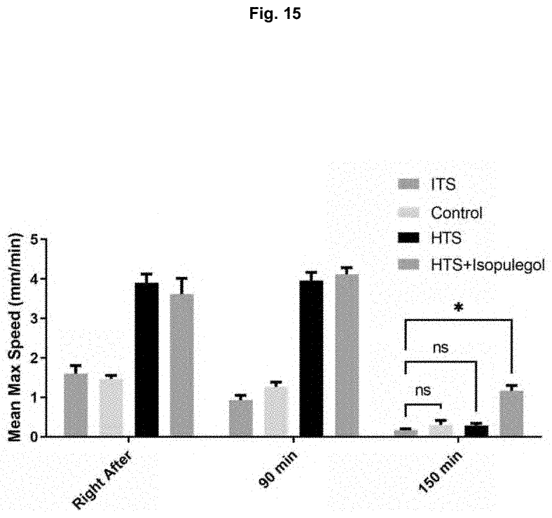

Similarly, FIG. 15 shows that the mucociliary clearance in ex vivo swine trachea preparations following treatment with HTS plus exemplary neuronal agonist (−)-isopulegol. The mucociliary clearance mean speed was measured immediately after HTS nebulization and 90 min, and 150 min after treatment. Addition of exemplary neuronal agonists (−)-isopulegol to the HTS formulation extended the duration of HTS-triggered mucociliary clearance in wild-type swine.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Donaldson, S. H. et al. Mucus clearance and lung function in cystic fibrosis with hypertonic saline. N. Engl. J. Med. 354, 241-250 (2006).
2. Elkins, M. R. & Bye, P. T. Inhaled hypertonic saline as a therapy for cystic fibrosis. Curr. Opin. Pulm. Med. 12, 445-452 (2006).
3. Tildy, B. E. & Rogers, D. F. Therapeutic options for hydrating airway mucus in cystic fibrosis. Pharmacology 95, 117-132 (2015).
4. Reeves, E. P. et al. Inhaled hypertonic saline for cystic fibrosis: Reviewing the potential evidence for modulation of neutrophil signalling and function. World J. Crit. Care Med. 4, 179-191 (2015).
5. Ellemunter, H., Eder, J., Fuchs, S., Gappa, M. & Steinkamp, G. Long-term improvement of lung clearance index in patients with mild cystic fibrosis lung disease: Does hypertonic saline play a role? J. Cyst. Fibros. 15, 123-126 (2016).
6. Ramsey, B. W. et al. A CFTR potentiator in patients with cystic fibrosis and the G551D mutation. N. Engl. J. Med. 365, 1663-1672 (2011).
7. Wainwright, C. E. et al. Lumacaftor-Ivacaftor in Patients with Cystic Fibrosis Homozygous for Phe508del CFTR. N. Engl. J. Med. 373, 220-231 (2015).
8. Taylor-Cousar, J. L. et al. Tezacaftor-Ivacaftor in Patients with Cystic Fibrosis Homozygous for Phe508del. N. Engl. J. Med. 377, 2013-2023 (2017).
9. Rowe, S. M. et al. Tezacaftor-Ivacaftor in Residual-Function Heterozygotes with Cystic Fibrosis. N. Engl. J. Med. 377, 2024-2035 (2017).
10. Bennett, W. D. et al. Duration of action of hypertonic saline on mucociliary clearance in the normal lung. J. Appl. Physiol. (1985) 118, 1483-1490 (2015).
11. Tarran, R. et al. The CF salt controversy: in vivo observations and therapeutic approaches. Mol. Cell 8, 149-158 (2001).
12. Goralski, J. L., Wu, D., Thelin, W. R., Boucher, R. C. & Button, B. The in vitro effect of nebulised hypertonic saline on human bronchial epithelium. Eur. Respir. J. 51, 10.1183/13993003.02652-2017 (2018).
13. Umeno, E., McDonald, D. M. & Nadel, J. A. Hypertonic saline increases vascular permeability in the rat trachea by producing neurogenic inflammation. J. Clin. Invest. 85, 1905-1908 (1990).
14. Barnes, P. J. Neurogenic inflammation in the airways. Respir. Physiol. 125, 145-154 (2001).
15. Maggi, C. A., Giachetti, A., Dey, R. D. & Said, S. I. Neuropeptides as regulators of airway function: vasoactive intestinal peptide and the tachykinins. Physiol. Rev. 75, 277-322 (1995).
16. Pedersen, K. E., Meeker, S. N., Riccio, M. M. & Undem, B. J. Selective stimulation of jugular ganglion afferent neurons in guinea pig airways by hypertonic saline. J. Appl. Physiol. (1985) 84, 499-506 (1998).
17. Fox, A. J., Barnes, P. J. & Dray, A. Stimulation of guinea-pig tracheal afferent fibres by non-isosmotic and low-chloride stimuli and the effect of frusemide. J. Physiol. 482 (Pt 1), 179-187 (1995).
18. Baraniuk, J. N., Ali, M., Yuta, A., Fang, S. Y. & Naranch, K. Hypertonic saline nasal provocation stimulates nociceptive nerves, substance P release, and glandular mucous exocytosis in normal humans. Am. J. Respir. Crit. Care Med. 160, 655-662 (1999).
19. Chou, Y. L., Scarupa, M. D., Mori, N. & Canning, B. J. Differential effects of airway afferent nerve subtypes on cough and respiration in anesthetized guinea pigs. Am. J. Physiol. Regul. Integr. Comp. Physiol. 295, R1572-1584 (2008).
20. Widdicombe, J. H. & Wine, J. J. Airway Gland Structure and Function. Physiol. Rev. 95, 1241-1319 (2015).
21. Ianowski, J. P., Choi, J. Y., Wine, J. J. & Hanrahan, J. W. Substance P stimulates CFTR-dependent fluid secretion by mouse tracheal submucosal glands. Pflugers. Arch. 457, 529-537 (2008).
22. Kishioka, C., Okamoto, K., Kim, J. S. & Rubin, B. K. Hyperosmolar solutions stimulate mucus secretion in the ferret trachea. Chest 124, 306-313 (2003).
23. Luan, X. et al. Pseudomonas aeruginosa triggers CFTR-mediated airway surface liquid secretion in swine trachea. Proc. Natl. Acad. Sci. USA 111, 12930-12935 (2014).
24. Luan, X. et al. Cystic fibrosis swine fail to secrete airway surface liquid in response to inhalation of pathogens. Nat. Commun. 8, 786, 10.1038/s41467-017-00835-7 (2017).
25. Morgan, K. S. et al. In vivo X-ray imaging reveals improved airway surface hydration after a therapy designed for cystic fibrosis. Am. J. Respir. Crit. Care Med. 190, 469-471 (2014).
26. Donaldson, S. H. & Boucher, R. C. Sodium channels and cystic fibrosis. Chest 132, 1631-1636 (2007).
27. Ajadi, A. R. et al. Tramadol improved the efficacy of ketamine-xylazine anaesthesia in young pigs. Vet. Anaesth. Analg. 36, 562-566 (2009).
28. Choudry, N. B., Fuller, R. W., Anderson, N. & Karlsson, J. A. Separation of cough and reflex bronchoconstriction by inhaled local anaesthetics. Eur. Respir. J. 3, 579-583 (1990).
29. Garland, A. et al. Hypertonicity, but not hypothermia, elicits substance P release from rat C-fiber neurons in primary culture. J. Clin. Invest. 95, 2359-2366 (1995).
30. Matera, M. G. et al. Evidence for non-adrenergic non-cholinergic contractile responses in bovine and swine trachea. Pulm. Pharmacol. Ther. 10, 105-110 (1997).
31. Scholz, A., Kuboyama, N., Hempelmann, G. & Vogel, W. Complex blockade of TTX-resistant Na+ currents by lidocaine and bupivacaine reduce firing frequency in DRG neurons. J. Neurophysiol. 79, 1746-1754 (1998).
32. Brau, M. E., Branitzki, P., Olschewski, A., Vogel, W. & Hempelmann, G. Block of neuronal tetrodotoxin-resistant Na+ currents by stereoisomers of piperidine local anesthetics. Anesth. Analg. 91, 1499-1505 (2000).
33. Wine, J. J. Parasympathetic control of airway submucosal glands: central reflexes and the airway intrinsic nervous system. Auton Neurosci 133, 35-54 (2007).
34. Melis, N. et al. Revisiting CFTR inhibition: a comparative study of CFTRinh-172 and GlyH-101 inhibitors. Br. J. Pharmacol. 171, 3716-3727 (2014).
35. Robinson, M. et al. Effect of hypertonic saline, amiloride, and cough on mucociliary clearance in patients with cystic fibrosis. Am. J. Respir. Crit. Care Med. 153, 1503-1509 (1996).
36. Robinson, M. et al. Effect of increasing doses of hypertonic saline on mucociliary clearance in patients with cystic fibrosis. Thorax 52, 900-903 (1997).
37. Choi, J. Y. et al. Substance P stimulates human airway submucosal gland secretion mainly via a CFTR-dependent process. J. Clin. Invest. 119, 1189-1200 (2009).
38. Khansaheb, M. et al. Properties of substance P-stimulated mucus secretion from porcine tracheal submucosal glands. Am. J. Physiol. Lung Cell Mol. Physiol. 300, L370-379 (2011).
39. Wine, J. J. & Joo, N. S. Submucosal glands and airway defense. Proc. Am. Thorac. Soc. 1, 47-53 (2004).
40. Joo, N. S., Saenz, Y., Krouse, M. E. & Wine, J. J. Mucus secretion from single submucosal glands of pig. Stimulation by carbachol and vasoactive intestinal peptide. J. Biol. Chem. 277, 28167-28175 (2002).
41. Joo, N. S., Krouse, M. E., Choi, J. Y., Cho, H. J. & Wine, J. J. Inhibition of airway surface fluid absorption by cholinergic stimulation. Sci. Rep. 6, 20735, 10.1038/srep20735 (2016).
42. Wine, J. J. et al. Measurement of fluid secretion from intact airway submucosal glands. Methods Mol. Biol. 742, 93-112 (2011).
43. Donnelley, M. et al. Non-invasive airway health assessment: synchrotron imaging reveals effects of rehydrating treatments on mucociliary transit in-vivo. Sci. Rep. 4, 3689, 10.1038/srep03689 (2014).
44. Morgan, K. S. et al. Measuring airway surface liquid depth in ex vivo mouse airways by x-ray imaging for the assessment of cystic fibrosis airway therapies. PLoS One 8, e55822, 10.1371/journal. pone.0055822 (2013).
45. Luan, X. et al. Nebulized hypertonic saline triggers nervous system-me diated active liquid secretion in cystic fibrosis swine trachea, Scientific Reports volume 9, Article number: 540 (2019).
46. Elkins M R, Robinson M, Rose B R, Harbour C, Moriarty C P, Marks G B, Belousova E G, Xuan W, Bye P T, and National Hypertonic Saline in Cystic Fibrosis Study G. A controlled trial of long-term inhaled hypertonic saline in patients with cystic fibrosis. N Engl J Med 354: 229-240, 2006.
47. Ellemunter H, Eder J, Fuchs S, Gappa M, and Steinkamp G. Long-term improvement of lung clearance index in patients with mild cystic fibrosis lung disease: Does hypertonic saline play a role? J Cyst Fibros 15: 123-126, 2016.
48 Reeves E P, McCarthy C, McElvaney O J, Vijayan M S, White M M, Dunlea D M, Pohl K, Lacey N, and McElvaney N G. Inhaled hypertonic saline for cystic fibrosis: Reviewing the potential evidence for modulation of neutrophil signalling and function. World J Crit Care Med 4: 179-191, 2015.
49 Rosenfeld M, Ratjen F, Brumback L, Daniel S, Rowbotham R, McNamara S, Johnson R, Kronmal R, Davis S D, and Group I S. Inhaled hypertonic saline in infants and children younger than 6 years with cystic fibrosis: the ISIS randomized controlled trial. JAMA 307: 2269-2277, 2012.
50. Tildy B E, and Rogers D F. Therapeutic options for hydrating airway mucus in cystic fibrosis. Pharmacology 95: 117-132, 2015.
51 Ratjen F, Davis S D, Stanojevic S, Kronmal R A, Hinckley Stukovsky K D, Jorgensen N, Rosenfeld M, and Group S S. Inhaled hypertonic saline in preschool children with cystic fibrosis (SHIP): a multicentre, randomised, double-blind, placebo-controlled trial. Lancet Respir Med 7: 802-809, 2019

1. A method of increasing the volume of an airway surface liquid (ASL) layer) or treating a disease, disorder or condition that is treatable by increasing the volume of an ASL layer comprising administering an effective amount of one or more neuronal agonists or a pharmaceutically acceptable salt, prodrug and/or solvate thereof, in combination with hypertonic saline (HTS) treatment or isotonic saline (ITS) treatment, to a subject in need thereof.

2. The method of claim 1 wherein the subject has cystic fibrosis and/or the subject has one or more non-cystic fibrosis respiratory diseases, disorders or conditions.

3. The method of claim 1, wherein the disease, disorder or condition is selected from cystic fibrosis and one or more non-cystic fibrosis respiratory diseases, disorders or conditions, and combinations thereof.

4. The method of claim 1, wherein the disease is cystic fibrosis.

5. The method of claim 1, wherein the disease, disorder or condition is selected from one or more non-cystic fibrosis respiratory fibrosis diseases, disorders or conditions and the one or more non-cystic fibrosis respiratory diseases, disorders or conditions are selected from one or more of non-cystic fibrosis bronchiectasis, pneumonia, obstructive pulmonary disease (COPD), asthma, bronchiolitis, bronchitis, mucoid impaction and primary ciliary dyskinesia.

6. The method of claim 1, wherein the disease, disorder or condition is selected from one or more respiratory non-cystic fibrosis respiratory diseases, disorders or conditions and the one or more non-cystic fibrosis respiratory diseases, disorders or conditions is a disease, disorder or condition characterized by an accumulation of and/or a difficulty in clearing of airway secretions arising from a neuromuscular disease, disorder or condition and/or rhinosinusitis.

7. The method of claim 1, wherein the one or more neuronal agonists are selected from one or more of capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol, isomenthol, carveol, terpineols, pulegol, isopulolegol, hinokitiol, myrtenol, and verbenol.

8. The method of claim 1, wherein the one or more neuronal agonists are selected from one or more of capsaicin, arvanil, olvanil, menthol and isopulolegol.

9. A method of increasing airway clearance or treating a disease, disorder or condition that is treatable by increasing airway clearance comprising administering an effective mount of one or more neuronal agonists, or a pharmaceutically acceptable salt, prodrug and/or solvate thereof, in combination with hypertonic saline (HTS) treatment or isotonic saline (ITS) treatment, to a subject in need thereof.

10. A method of increasing mucociliary clearance or treating a disease, disorder or condition that is treatable by increasing mucociliary clearance comprising administering an effective amount of one or more neuronal agonists, or a pharmaceutically acceptable salt, prodrug and/or solvate thereof, in combination with hypertonic saline (HTS) treatment or isotonic saline (ITS) treatment, to a subject in need thereof.

11. The method of claim 10, wherein the subject has cystic fibrosis and/or the subject has one or more non-cystic fibrosis respiratory diseases, disorders or conditions.

12. The method of claim 10, wherein the subject has one or more non-cystic fibrosis respiratory diseases, disorders or conditions.

13. The method of claim 10, wherein the disease, disorder or condition is selected from cystic fibrosis and one or more non-cystic fibrosis respiratory diseases, disorders or conditions and combinations thereof.

14. The method of claim 10, wherein the disease, disorder or condition is selected from one or more non-cystic fibrosis respiratory diseases, disorders or conditions and the one or more non-cystic fibrosis respiratory diseases, disorders or conditions are selected from one or more of non-cystic fibrosis bronchiectasis, pneumonia, obstructive pulmonary disease (COPD), asthma, bronchiolitis, bronchitis, mucoid impaction and primary ciliary dyskinesia.

15. The method of claim 10, wherein the disease, disorder or condition is selected from one or more respiratory non-cystic fibrosis diseases, disorders or conditions and the one or more non-cystic fibrosis respiratory diseases, disorders or conditions is a disease, disorder or condition characterized by an accumulation of and/or a difficulty in clearing of airway secretions arising from a neuromuscular disease, disorder or condition and/or rhinosinusitis.

16. The method of claim 10, wherein the one or more neuronal agonists are selected from capsaicin, dihydrocapsaicin, nordihydrocapsaicin, homocapsaicin, homodihydrocapsaicin, noivamide, capsiate, dihydrocapsiate, nordihydrocapsiate, arvanil, olvanil, menthol, isomenthol, carveol, terpineols, pulegol, isopulolegol, hinokitiol, myrtenol, and verbenol and combinations thereof.

17. The method of claim 10, wherein the one or more neuronal agonists are selected from capsaicin, arvanil, olvanil, menthol and isopulolegol and combinations thereof.

18. A method of improving the efficacy of hypertonic saline (HTS) treatment or isotonic saline (ITS) treatment comprising administering an effective amount of one or more neuronal agonists or a pharmaceutically acceptable salt, prodrug and/or solvate thereof, in combination with the HTS treatment, or ITS treatment to a subject in need thereof.

19. A composition comprising one or more neuronal agonists, or a pharmaceutically acceptable salt, prodrug and/or solvate thereof, and hypertonic saline (HTS) or isotonic saline (ITS), wherein the one or more neuronal agonists, or a pharmaceutically acceptable salt, prodrug and/or solvate thereof, and HTS or ITS are present in amounts effective to increase mucociliary clearance or for treating a disease, disorder or condition treatable by increasing mucociliary clearance.

20. The composition of claim 19, wherein the one or more neuronal agonists, or a pharmaceutically acceptable salt, prodrug and/or solvate thereof are formulated for administration by inhalation and/or the HTS or ITS is formulated for administration by inhalation.
